SEQUENCE LISTING (23 sequences)

SEQ ID NO: 1 - protein, 7 aa, Unknown
  source: /note=“Description of Unknown Angiotensin (1-7) sequence”
DRVYIHP

SEQ ID NO: 2 - protein, 7 aa, Artificial Sequence
  source: /note=“Description of Artificial Sequence Synthetic peptide”
DRVSIHC

SEQ ID NO: 3 - protein, 7 aa, Artificial Sequence
  source: /note=“Description of Artificial Sequence Synthetic peptide”
ARVSIHC

SEQ ID NO: 4 - protein, 7 aa, Artificial Sequence
  source: /note=“Description of Artificial Sequence Synthetic peptide”
  MOD_RES (1)..(1): Any amino acid or a dicarboxylic acid
  VARIANT (2)..(2): /replace=“Lys” or “Ala” or “Cit” or “Orn” or “acSer” or “Sar” or “D-Arg” or “D-Lys”
  VARIANT (3)..(3): /replace=“Ala” or “Leu” or “Nle” or “Ile” or “Gly” or “Lys” or “Pro” or “HydroxyPro” or “Aib” or “Acpc” or “Tyr”
  VARIANT (4)..(4): /replace=“Tyr(PO3)” or “Thr” or “Ser” or “homoSer” or “azaTyr” or “Ala”
  VARIANT (5)..(5): /replace=“Ala” or “Leu” or “Nle” or “Val” or “Gly”
  VARIANT (6)..(6): /replace=“Arg” or “6-NH2-Phe”
  VARIANT (7)..(7): /replace=“Pro” or “Ala”
  SITE (1)..(7): /note=“Variant residues given in the sequence have no preference with respect to those in the annotations for variant positions”
  source: /note=“See specification as filed for detailed description of substitutions and preferred embodiments”
XRVYIHC

SEQ ID NO: 5 - protein, 7 aa, Artificial Sequence
  source: /note=“Description of Artificial Sequence Synthetic peptide”
  MOD_RES (3)..(3): Nle
DRXYIHP

SEQ ID NO: 6 - protein, 7 aa, Artificial Sequence
  source: /note=“Description of Artificial Sequence Synthetic peptide”
DRVSIHC

SEQ ID NO: 7 - protein, 7 aa, Artificial Sequence
  source: /note=“Description of Artificial Sequence Synthetic peptide”
  MOD_RES (4)..(4): Any amino acid forming a thioether bridge
  SITE (4)..(7): /note=“Thioether bridge”
  MOD_RES (7)..(7): Any amino acid forming a thioether bridge
DRVXIHX

SEQ ID NO: 8 - protein, 10 aa, Artificial Sequence
  source: /note=“Description of Artificial Sequence Synthetic peptide”
  MOD_RES (3)..(3): Nle
  MOD_RES (4)..(4): Any amino acid forming a thioether bridge
  SITE (4)..(7): /note=“Thioether bridge”
  MOD_RES (7)..(7): Any amino acid forming a thioether bridge
  source: /note=“See specification as filed for detailed description of substitutions and preferred embodiments”
DRXXIHXFHL

SEQ ID NO: 9 - protein, 8 aa, Artificial Sequence
  source: /note=“Description of Artificial Sequence Synthetic peptide”
  MOD_RES (3)..(3): Nle
  MOD_RES (4)..(4): Any amino acid forming a thioether bridge
  SITE (4)..(7): /note=“Thioether bridge”
  MOD_RES (7)..(7): Any amino acid forming a thioether bridge
  source: /note=“See specification as filed for detailed description of substitutions and preferred embodiments”
DRXXIHXF

SEQ ID NO: 10 - protein, 7 aa, Artificial Sequence
  source: /note=“Description of Artificial Sequence Synthetic peptide”
  MOD_RES (2)..(2): Nle
  MOD_RES (3)..(3): Any amino acid forming a thioether bridge
  SITE (3)..(6): /note=“Thioether bridge”
  MOD_RES (6)..(6): Any amino acid forming a thioether bridge
  source: /note=“See specification as filed for detailed description of substitutions and preferred embodiments”
RXXIHXF

SEQ ID NO: 11 - protein, 6 aa, Artificial Sequence
  source: /note=“Description of Artificial Sequence Synthetic peptide”
  MOD_RES (1)..(1): Nle
  MOD_RES (2)..(2): Any amino acid forming a thioether bridge
  SITE (2)..(5): /note=“Thioether bridge”
  MOD_RES (5)..(5): Any amino acid forming a thioether bridge
  source: /note=“See specification as filed for detailed description of substitutions and preferred embodiments”
XXIHXF

SEQ ID NO: 12 - protein, 7 aa, Artificial Sequence
  source: /note=“Description of Artificial Sequence Synthetic peptide”
  MOD_RES (3)..(3): Nle
  MOD_RES (4)..(4): Any amino acid forming a thioether bridge
  SITE (4)..(7): /note=“Thioether bridge”
  MOD_RES (7)..(7): Any amino acid forming a thioether bridge
  source: /note=“See specification as filed for detailed description of substitutions and preferred embodiments”
DRXXIHX

SEQ ID NO: 13 - protein, 9 aa, Artificial Sequence
  source: /note=“Description of Artificial Sequence Synthetic peptide”
  MOD_RES (3)..(3): Nle
  MOD_RES (4)..(4): Any amino acid forming a thioether bridge
  SITE (4)..(7): /note=“Thioether bridge”
  MOD_RES (7)..(7): Any amino acid forming a thioether bridge
  source: /note=“See specification as filed for detailed description of substitutions and preferred embodiments”
DRXXIHXFH

SEQ ID NO: 14 - protein, 7 aa, Artificial Sequence
  source: /note=“Description of Artificial Sequence Synthetic peptide”
  MOD_RES (1)..(1): Any amino acid
  MOD_RES (2)..(2): Any positively charged amino acid
  MOD_RES (3)..(3): Any aliphatic amino acid
  MOD_RES (4)..(4): Any amino acid forming a thioether bridge
  SITE (4)..(7): /note=“Thioether bridge”
  MOD_RES (5)..(5): Any aliphatic amino acid
  MOD_RES (7)..(7): Any amino acid forming a thioether bridge
  source: /note=“See specification as filed for detailed description of substitutions and preferred embodiments”
XXXXXHX

SEQ ID NO: 15 - protein, 7 aa, Artificial Sequence
  source: /note=“Description of Artificial Sequence Synthetic peptide”
  MOD_RES (4)..(4): Abu
DRVXIHA

SEQ ID NO: 16 - protein, 7 aa, Artificial Sequence
  source: /note=“Description of Artificial Sequence Synthetic peptide”
DRVAIHA

SEQ ID NO: 17 - protein, 10 aa, Artificial Sequence
  source: /note=“Description of Artificial Sequence Synthetic peptide”
  MOD_RES (1)..(1): Any amino acid or absent
  MOD_RES (2)..(2): Any positively charged amino acid or absent
  MOD_RES (3)..(3): Nle
  MOD_RES (4)..(4): Any amino acid forming a thioether bridge
  SITE (4)..(7): /note=“Thioether bridge”
  MOD_RES (5)..(5): Any aliphatic amino acid
  MOD_RES (7)..(7): Any amino acid forming a thioether bridge
  MOD_RES (8)..(8): Any amino acid other than Pro or is absent
  VARIANT (9)..(9): /replace=“ ”
  MOD_RES (10)..(10): Any aliphatic residue or absent
  SITE (1)..(10): /note=“Variant residues given in the sequence have no preference with respect to those in the annotations for variant positions”
  source: /note=“See specification as filed for detailed description of substitutions and preferred embodiments”
XXXXXHXXHX

SEQ ID NO: 18 - protein, 7 aa, Artificial Sequence
  source: /note=“Description of Artificial Sequence Synthetic peptide”
  MOD_RES (3)..(3): Nle
  MOD_RES (4)..(4): Abu
DRXXIHA

SEQ ID NO: 19 - protein, 7 aa, Artificial Sequence
  source: /note=“Description of Artificial Sequence Synthetic peptide”
  MOD_RES (3)..(3): Nle
DRXAIHA

SEQ ID NO: 20 - protein, 8 aa, Artificial Sequence
  source: /note=“Description of Artificial Sequence Synthetic peptide”
  MOD_RES (3)..(3): Nle
  MOD_RES (4)..(4): Abu
DRXXIHAI

SEQ ID NO: 21 - protein, 8 aa, Artificial Sequence
  source: /note=“Description of Artificial Sequence Synthetic peptide”
  MOD_RES (3)..(3): Nle
DRXAIHAI

SEQ ID NO: 22 - protein, 7 aa, Artificial Sequence
  source: /note=“Description of Artificial Sequence Synthetic peptide”
DRVSIHC

SEQ ID NO: 23 - protein, 12 aa, Unknown
  source: /note=“Description of Unknown Angiotensin sequence”
DRVYIHPFHLVI

(19) United States
(12) Patent Application Publication
(10) Pub. No.: US 2023/0110554 A1
(43) Pub. Date: Apr. 13, 2023

(54) METHODS AND COMPOSITIONS FOR TREATMENT OF CORONAVIRUS INFECTION

(71) Applicant: CONSTANT THERAPEUTICS LLC, Cambridge, MA (US)

(72) Inventor: Richard FRANKLIN, Cave Creek, AZ (US)

(21) Appl. No.: 17/911,233

(22) PCT Filed: Mar. 12, 2021
(86) PCT No.: PCT/US2021/022077

§ 371 Date: Sep. 13, 2022

Related U.S. Application Data
(60) Provisional application No. 62/989,145, filed on Mar. 13, 2020.

(51) Int. Cl. A61K 38/08; A61K 31/64; A61P 31/14; A61P 11/00

(52) U.S. Cl. CPC A61K 38/085; A61K 31/64; A61P 31/14; A61P 11/00

(57) ABSTRACT

The present invention provides methods and compositions for treating lung disease in individuals as a complication of infection by pathogens, including coronaviruses. The present invention is based on the use of an angiotensin (1-7) peptide or angiotensin (1-7) receptor agonist in treating patients infected with coronaviruses including COVID-19, SARS, MERS and others. In some embodiments, the angiotensin (1-7) peptide or angiotensin (1-7) receptor agonist is used to treat lung diseases including but not limited to bronchitis, pneumonia, asthma, pulmonary fibrosis and acute respiratory distress syndrome.

### CROSS-REFERENCE TO RELATED APPLICATIONS

This application claims benefit of, and priority to, U.S. Ser. No. 62/989,145 filed on Mar. 13, 2020, the contents of which is hereby incorporated by reference in its entirety.

### BACKGROUND

Coronaviruses (CoVs) primarily target mucosal epithelia of respiratory and intestinal tracts resulting in respiratory and enteric symptoms. Although most infections result in mild self-limiting symptoms such as the common cold, novel emerging strains have been the causative agents of global outbreaks with significant health and economic impacts such as Severe Acute Respiratory Syndrome (SARS), Middle East Respiratory Syndrome (MERS) and Coronavirus Disease 2019 (COVID-19).

Coronavirus spread is extremely rapid and complications associated with acute and severe respiratory disease can occur in elderly and immunocompromised adults. Injury to the lung, in particular, can have dramatic and potentially fatal effects.

No vaccine is available, and control measures are limited to supportive care and preventing spread by isolation and containment.

### SUMMARY OF THE INVENTION

The present invention provides, among other things, methods and compositions for treating individuals infected with a coronavirus, such that one or more symptoms or complications of the coronavirus infection is reduced in intensity, severity, duration, or frequency or has delayed onset. In particular, it is contemplated that angiotensin (1-7) peptides or angiotensin (1-7) receptor agonists can be used in treatment of lung disease or conditions, including bronchitis, pulmonary fibrosis, asthma, acute respiratory distress syndrome, and pneumonia that results as a complication of infection by viruses including coronaviruses COVID-19, SARS and MERS, respiratory syncytial virus, avian influenza A H5N1, H1N1 virus and others, as well as other pathogens.

In one aspect, the present application provides a method of treating a subject infected with a coronavirus comprising administering to the subject an angiotensin (1 7) peptide or angiotensin (1-7) receptor agonist.

In some embodiments, the subject infected with a coronavirus is infected with COVID-19, SARS or MERS.

In some embodiments, the angiotensin (1-7) peptide is administered at an effective dose periodically at an administration interval such that one or more symptoms or complications of the coronavirus infection is reduced in intensity, severity, duration, or frequency or has delayed onset.

In some embodiments, the one or more symptoms or complications of the coronavirus infection are selected from flu-like symptoms, fever, cough, myalgia, fatigue, pneumonia, or breathing difficulties. In some embodiments, the one or more symptoms or complications of the coronavirus infection comprise a cytokine storm.

In one aspect, the present application provides a method of treating a lung disease or condition comprising administering to the subject in need of treatment an angiotensin (1-7) peptide or angiotensin (1-7) receptor agonist.

In some embodiments, the lung disease or condition is selected from bronchitis, pneumonia, pulmonary fibrosis, asthma or acute respiratory distress syndrome.

In some embodiments, the lung disease or condition is caused by a coronavirus infection.

In some embodiments, the coronavirus infection is COVID-19, SARS or MERS.

In some embodiments, the lung disease or condition is caused by avian flu H5N1. In some embodiments, the lung disease or condition is caused by H1N1. In some embodiments, the lung disease or condition is caused by RSV.

In some embodiments, the administration is parenteral, rectal, oral, or a combination thereof. In some embodiments, the parenteral administration is intravenous, subcutaneous, inhalation, intradermal, transdermal, and/or transmucosal administration. In some embodiments, the administration is oral. In some, embodiments Ang-(1-7) is formulated with cyclodextrin for oral administration.

In some embodiments, the angiotensin (1-7) peptide comprises an amino acid sequence of Asp¹-Arg²-Val³-Tyr⁴-Ile⁵-His⁶-Pro⁷ (SEQ ID NO: 1) or a functional equivalent thereof. In some embodiments, the angiotensin (1-7) peptide is a linear peptide.

In some embodiments, the angiotensin (1-7) peptide has SEQ ID NO: 1.

In some embodiments, the angiotensin (1-7) peptide comprises an amino acid sequence of Asp¹-Arg²-Val³-Ser⁴-Ile⁵-His⁶-Cys⁷ (SEQ ID NO: 2) or a functional equivalent thereof.

In some embodiments, the angiotensin (1-7) peptide has an amino acid sequence of Ala¹-Arg²-Val³-Ser⁴-Ile⁵-His⁶-Cys⁷ (SEQ ID NO: 3) or a functional equivalent thereof.

In some embodiments, the angiotensin (1-7) peptide comprises one or more chemical modifications to increase protease resistance, serum stability and/or bioavailability.

In some embodiments, the one or more chemical modifications comprise pegylation.

In some embodiments, the angiotensin (1-7) peptide or angiotensin (1-7) receptor agonist is administered in combination with another treatment of the coronavirus infection or another treatment of lung disease or condition.

In some embodiments, the angiotensin (1-7) peptide is administered at an effective dose ranging from about 1-1,000 m/kg/day.

In some embodiments, the angiotensin (1-7) receptor agonist is a compound with the following structure:

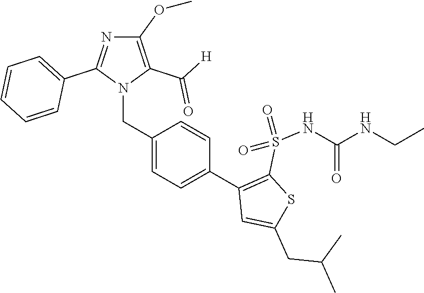 [SMILES: CCNC(=O)NS(=O)(=O)c1sc(CC(C)C)cc1-c1ccc(Cn2c(-c3ccccc3)nc(OC)c2C=O)cc1]

or a pharmaceutically acceptable salt thereof.

### Definitions

In order for the present invention to be more readily understood, certain terms are first defined below. Additional definitions for the following terms and other terms are set forth throughout the specification. In order for the present invention to be more readily understood, certain terms are first defined below. Additional definitions for the following terms and other terms are set forth throughout the specification.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion. In certain embodiments, a peptide has no intrinsic biological activity but that inhibits the effects of one or more naturally-occurring angiotensin compounds is considered to be biologically active.

Carrier or diluent: As used herein, the terms “carrier” and “diluent” refers to a pharmaceutically acceptable (e.g., safe and non-toxic for administration to a human) carrier ordiluting substance useful for the preparation of a pharmaceutical formulation. Exemplary diluents include sterile water, bacteriostatic water for injection (BWFI), a pH buffered solution(e.g. phosphate-buffered saline), sterile saline solution, Ringer's solution or dextrose solution.

Complication: As used herein, the term “complication” refers to an unfavorable evolution of a disease including the development of one or more signs, symptoms or, in some embodiments, even new pathological changes that manifest for a sustained period of time (e.g., weeks, months or years). In some embodiments, complication(s) may include a progression of a sign, symptom or other pathological change, for example, breathing difficulties growing worse over time, or a difficulty with one or more respiratory functions progressing to acute respiratory distress.

Dosage form: As used herein, the terms “dosage form” and “unit dosage form” refer to a physically discrete unit of a therapeutic agent for the patient to be treated. Each unit contains a predetermined quantity of active material calculated to produce the desired therapeutic effect. It will be understood, however, that the total dosage of the composition will be decided by the attending physician within the scope of sound medical judgment.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regime comprises a plurality of doses and at least two different time periods separating individual doses. In some embodiments, the therapeutic agent is administered continuously over a predetermined period. In some embodiments, the therapeutic agent is administered once a day (QD) or twice a day (BID).

Functional equivalent or derivative: As used herein, the term “functional equivalent” or “functional derivative” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism.

Prevent: As used herein, the term “prevent” or “prevention”, when used in connection with the occurrence of a disease, disorder, and/or condition, refers to reducing the risk of developing the disease, disorder and/or condition. See the definition of “risk.”

Polypeptide: The term “polypeptide” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Risk: As will be understood from context, a “risk” of a disease, disorder, and/or condition comprises a likelihood that a particular individual will develop a disease, disorder, and/or condition (e.g., lung injury). In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event (e.g., lung injury). In some embodiments a reference sample or group of reference samples are from individuals comparable to a particular individual. In some embodiments, relative risk is 0, 1, 2, 3, 4, 5, 6, 7, 8, 9, 10, or more.

Stability: As used herein, the term “stable” refers to the ability of the therapeutic agent to maintain its therapeutic efficacy (e.g., all or the majority of its intended biological activity and/or physiochemical integrity) over extended periods of time. The stability of a therapeutic agent, and the capability of the pharmaceutical composition to maintain stability of such therapeutic agent, may be assessed over extended periods of time (e.g., for at least 1, 3, 6, 12, 18, 24, 30, 36 months or more). In certain embodiments, pharmaceutical compositions described herein have been formulated such that they are capable of stabilizing, or alternatively slowing or preventing the degradation, of one or more therapeutic agents formulated therewith. In the context of a formulation a stable formulation is one in which the therapeutic agent therein essentially retains its physical and/or chemical integrity and biological activity upon storage and during processes (such as freeze/thaw, mechanical mixing and lyophilization).

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of the disease, disorder, and/or condition.

Susceptible to: An individual who is “susceptible to” a disease, disorder, and/or condition has not been diagnosed with the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, condition, or event (for example, lung injury) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein associated with the disease, disorder, and/or condition; (4) habits and/or lifestyles associated with development of the disease, disorder, condition, and/or event (5) having undergone, planning to undergo, or requiring a transplant. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### BRIEF DESCRIPTION OF THE DRAWING

Drawings are for illustration purposes only; not for limitation.

FIG. 1 shows an exemplary illustration of the renin-angiotensin (RAS) system. In the classical pathway, Angiotensin Converting Enzyme (ACE) cleaves Angiotensinogen (Ang) I to Angiotensinogen II which promotes lung injury through the AT1R signaling that leads to vasoconstriction and inflammation. Angiotensin Converting Enzyme 2 (ACE2) cleaves Angiotensinogen II to Angiotensin (1-7) in the alternative renin-angiotensin pathway that exerts a protective effect against lung injury. Coronavirus infection downregulates ACE2 and promotes lung injury.

### DETAILED DESCRIPTION

Among other things, the present application provides a method of treating a subject infected with a coronavirus by administering to the subject an angiotensin (1-7) peptide or angiotensin (1-7) receptor agonist. The present invention further provides a method of treating a lung disease or condition, particularly caused by a virus infection, by administering to the subject in need of treatment an angiotensin (1-7) peptide or angiotensin (1-7) receptor agonist.

### Coronavirus Infection

Coronaviruses (CoVs) are enveloped viruses with a positive-strand RNA genome, which primarily target respiratory and intestinal mucosal surfaces to establish respiratory and enteric infections. Their name derives from their crown-like appearance in electron micrographs.

Coronaviruses such as the human CoV isolates 229E and OC43 cause mild and self-limiting infections of the respiratory tract such as the common cold. Novel isolates HCoV-NL63 and HCoV-HKU1 have also been associated with common cold.

Some novel emerging coronaviruses have resulted in serious global outbreaks such as SARS in 2003, MERS in 2012 and COVID-19 in 2019. These diseases are associated with acute and often severe respiratory complications, especially in elderly and immunocompromised individuals.

COVID-19 infection results in respiratory symptoms like a runny nose, headache, dry cough, sore throat, shortness of breath, fever and fatigue. Symptoms of COVID-19 may appear 2-14 days after exposure. Older adults and those with underlying health conditions and weakened immune systems may be at risk for more severe symptoms.

Once COVID-19 enters the human respiratory tract, it infects and propagates in cells lining the airway, causing damage that triggers local inflammation, recruiting immune cells in the vicinity to eradicate the virus. The immune response then recedes, and patients recover from mild disease. In elderly and other vulnerable individuals, there is instead an uncontrolled immune response, triggering an overproduction of immune cells and signaling molecules, leading to a cytokine storm often associated with a flood of immune cells into the lung. If an infection progresses to something more severe, it can cause bronchitis, pneumonia, kidney failure and even death.

Pneumonia associated with COVID-19 usually has bilateral lung involvement. Pneumonia is characterized by inflammation of the alveoli or air sacs, which may fill with fluid or pus (purulent material), causing cough with phlegm or pus, fever, chills, and difficulty breathing. Pneumonia may cause pleural effusion, i.e. fluid accumulation in the thin space between layers of tissue that line the lungs and chest cavity (pleura), which may lead to pus accumulation, infection and lung abscess. In severe cases, lung fibrosis may occur. Pulmonary fibrosis occurs when the lung tissue around and between the alveoli becomes thickened, damaged and scarred, making it more difficult for oxygen to pass into the bloodstream. This is accompanied by fibroblast proliferation and an increase in matrix proteins, abnormal alveolar structure leading to loss of lung function and severe respiratory failure. Some individuals may experience a rapid worsening of their symptoms (acute exacerbation), such as severe shortness of breath, that may last for several days to weeks. Individuals with acute exacerbations may be placed on a mechanical ventilator. Pulmonary fibrosis is usually irreversible and is managed by corticosteroid therapy, but could potentially be fatal.

Acute respiratory distress syndrome (ARDS), the most severe form of acute lung injury (ALI), is a devastating clinical syndrome with high mortality rate (30-60%). ARDS is characterized by breakdown of the alveolar-capillary barrier, leading to flooding of the alveolar space. Predisposing factors for ARDS are diverse and include sepsis, aspiration, and pneumonias including infections with coronavirus, H1N1 and H5N1 avian influenza virus. Several other pathogens including Spanish flu virus and anthrax can result in ARDS. To date, no effective cure exists for ARDS.

SARS-CoV and COVID-19 rapidly spread person-to-person through respiratory secretions. Upon exposure of the host to the virus, the viral Spike protein (which projects through the viral envelope forming the characteristic spikes in the coronavirus), binds to epithelial cells expressing virus entry receptors, of which the angiotensin-converting enzyme 2 (ACE2) is one of the main receptors. In the respiratory tract, ACE2 is widely expressed on epithelial cells of alveoli, trachea, bronchi, bronchial serous glands, and alveolar monocytes and macrophages. The virus enters and replicates in these target cells, releasing mature virions which infect new target cells. Atypical pneumonia with rapid respiratory deterioration and lung injury can result due to increased levels of activated proinflammatory chemokines and cytokines.

Other pathogens including the influenza H5N1 and H1N1 viruses and RSV employ the ACE2 receptor for entry and the renin-angiotensin system plays a role in host response to infection.

### Renin-Angiotensin System

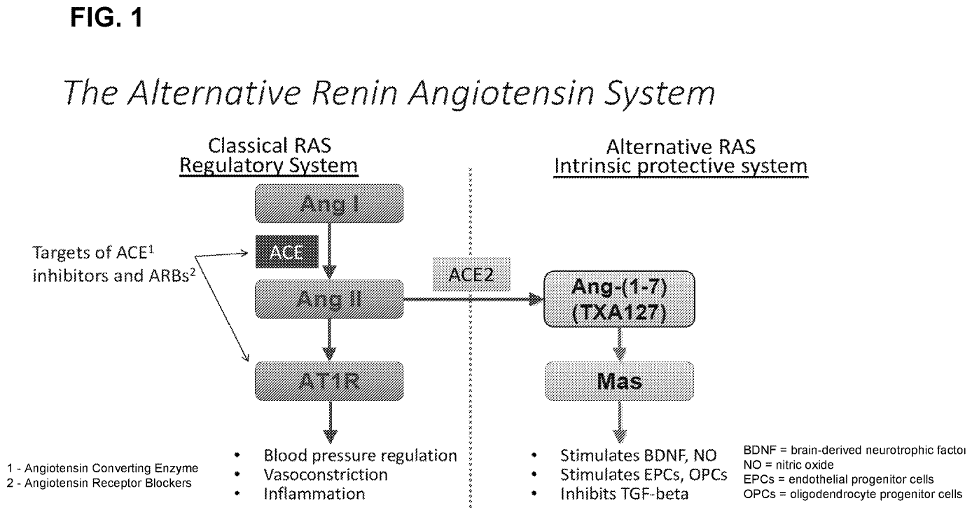

The renin-angiotensin system (RAS) maintains blood pressure and electrolyte balance in the body, important for cardiovascular and renal functions (FIG. 1). Renin is a protease that cleaves angiotensinogen to generate angiotensin (Ang) I. Angiotensin-converting enzyme (ACE) cleaves Ang I to produce Ang II, which binds Ang II receptor type 1(AT1R) and Ang II receptor type 2 (AT2R) to carry out various biological functions in blood pressure regulation, mediating vasoconstriction, proliferative, fibrotic and inflammatory effects (Skeggs et al. 1980; Corvol et al. 1995).

ACE2 is a homologue of ACE that cleaves the octapeptide Ang II to a heptapeptide Ang-(1-7), which acts via the G-protein coupled Mas receptor in the alternative renin-angiotensin pathway to oppose the vasoconstrictive and inflammatory effects of the classical pathway mediated by ACE/AngII/AT1R signaling (Douglas et al., 2004, Kuba et al., 2013, Jiang et al, 2014). Ang-(1-7) acts through a variety of downstream targets, including inhibition of fibrosis due to TGF-β, reduction of reactive oxygen species and oxidative stress, stimulation of brain derived neurotrophic factor (BDNF), activation of endothelial NO synthase (eNOS) and thus the release of NO, increasing vasodilation and endothelial protection. Ang-(1-7) functions in the stabilization of epithelial and endothelial membranes and stimulation of the proliferation and release of endothelial progenitor cells (EPCs) from the bone marrow increasing lung tissue repair and vascular remodeling. In addition, ACE2 functions to modulate innate immunity and influence the composition of gut microbiota.

Angiotensin II contributes to acute lung failure through stimulation of the AT1R, while ACE2 and AT2R negatively regulate this pathway and protect from acute lung failure.

ACE2 is found in alveolar epithelial cells, enterocytes in the small intestines and endothelial and smooth muscle cells in several organs. ACE2 and its product Ang-(1-7) intrinsically protect lungs from severe acute injury by blocking the classical renin-angiotensin pathway. ACE2 and Ang-(1-7) exert a protective role against a variety of pulmonary diseases, including but not limited to pulmonary fibrosis, acute lung injury, acute respiratory distress syndrome, pulmonary hypertension, asthma, and chronic obstructive pulmonary disease. In a coronavirus infection, the coronavirus spike protein downregulates ACE2 expression (Imai et al, 2005), promoting lung injury. Downregulation of ACE2 is associated with a reduction of its enzymatic product Ang-(1-7). In one aspect, this application provides a method of treating a subject infected with a coronavirus comprising administering to the subject an angiotensin (1-7) peptide or angiotensin (1-7) receptor agonist.

Various aspects of the invention are described in detail in the following sections. The use of sections is not meant to limit the invention. Each section can apply to any aspect of the invention. In this application, the use of “or” means “and/or” unless stated otherwise.

### Angiotensin (1-7)Peptides

As used herein, the term “angiotensin (1-7) peptide” refers to both naturally-occurring Angiotensin (1-7) and any functional equivalent, analogue or derivative of naturally-occurring Angiotensin (1-7). As used herein, “peptide” and “polypeptide” are interchangeable terms and refer to two or more amino acids bound together by a peptide bond. As used herein, the terms “peptide” and “polypeptide” include both linear and cyclic peptide. The terms “angiotensin-(1-7)”, “Angiotensin-(1-7)”, and “Ang-(1-7)” are used interchangeably.

### Naturally-Occurring Angiotensin (1-7)

Naturally-occurring Angiotensin (1-7) (also referred to as Ang-(1-7)) is a seven amino acid peptide shown below:

(SEQ ID NO: 1)
Asp¹-Arg²-Val³-Tyr⁴-Ile⁵-His⁶-Pro⁷

It is part of the renin-angiotensin system and is converted from a precursor, also known as Angiotensinogen, which is an α-2-globulin that is produced constitutively and released into the circulation mainly by the liver. Angiotensinogen is a member of the serpin family and also known as renin substrate. Human angiotensinogen is 452 amino acids long, but other species have angiotensinogen of varying sizes. Typically, the first 12 amino acids are the most important for angiotensin activity:

(SEQ ID NO: 23)
Asp¹-Arg²-Val³-Tyr⁴-Ile⁵-His⁶-Pro⁷-Phe⁸-His⁹-
Leu¹⁰-Val¹¹-Ile¹²

Different types of angiotensin may be formed by the action of various enzymes. For example, Angiotensin (1-7) is generated by action of Angiotensin-converting enzyme 2 (ACE 2).

Ang-(1-7) is an endogenous ligand for Mas receptors. Mas receptors are G-protein coupled receptor containing seven transmembrane spanning regions. As used herein, the term “angiotensin-(1-7) receptor” encompasses the G Protein-Coupled Mas Receptors.

As used herein, the term “naturally-occurring Angiotensin (1-7)” includes any Angiotensin (1-7) peptide purified from natural sources and any recombinantly produced or chemically synthesized peptides that have an amino acid sequence identical to that of the naturally-occurring Angiotensin (1-7).

### Functional Equivalents, Analogs or Derivatives of Ang-(1-7)

In some embodiments, an angiotensin (1-7) peptide suitable for the present invention is a functional equivalent of naturally-occurring Ang-(1-7). As used herein, a functional equivalent of naturally-occurring Ang-(1-7) refers to any peptide that shares amino acid sequence identity to the naturally-occurring Ang-(1-7) and retain substantially the same or similar activity as the naturally-occurring Ang-(1-7). For example, in some embodiments, a functional equivalent of naturally-occurring Ang-(1-7) described herein has pro-angiogenic activity as determined using methods described herein or known in the art, or an activity such as nitric oxide release, vasodilation, improved endothelial function, antidiuresis, or one of the other properties discussed herein, that positively impacts angiogenesis. In some embodiments, a functional equivalent of naturally-occurring Ang-(1-7) described herein can bind to or activate an angiotensin-(1-7) receptor (e.g., the G protein-coupled Mas receptor) as determined using various assays described herein or known in the art. In some embodiments, Ang-(1-7) inhibits fibrosis by downregulating TGF-β. In some embodiments, Ang-(1-7) reduces reactive oxygen species and oxidative stress. In some embodiments, Ang-(1-7) stimulates BDNF. In some embodiments, a functional equivalent of Ang-(1-7) is also referred to as an angiotensin (1-7) analogue or derivative, or functional derivative.

Typically, a functional equivalent of angiotensin (1-7) shares amino acid sequence similarity to the naturally-occurring Ang-(1-7). In some embodiments, a functional equivalent of Ang-(1-7) according to the invention contains a sequence that includes at least 3 (e.g., at least 4, at least 5, at least 6, at least 7) amino acids from the seven amino acids that appear in the naturally-occurring Ang-(1-7), wherein the at least 3 (e.g., at least 4, at least 5, at least 6, or at least 7) amino acids maintain their relative positions and/or spacing as they appear in the naturally-occurring Ang-(1-7).

In some embodiments, a functional equivalent of Ang-(1-7) also encompasses any peptide that contains a sequence at least 50% (e.g., at least 60%, 70%, 80%, or 90%) identical to the amino acid sequence of naturally-occurring Ang-(1-7). Percentage of amino acid sequence identity can be determined by alignment of amino acid sequences. Alignment of amino acid sequences can be achieved in various ways that are within the skill in the art, for instance, using publicly available computer software such as BLAST, ALIGN or Megalign (DNASTAR) software. Those skilled in the art can determine appropriate parameters for measuring alignment, including any algorithms needed to achieve maximal alignment over the full length of the sequences being compared. Preferably, the WU-BLAST-2 software is used to determine amino acid sequence identity (Altschul et al., Methods in Enzymology 266, 460-480 (1996); http://blast.wustl/edu/blast/README.html). WU-BLAST-2 uses several search parameters, most of which are set to the default values. The adjustable parameters are set with the following values: overlap span=1, overlap fraction=0.125, word threshold (T)=11. HSP score (S) and HSPS2 parameters are dynamic values and are established by the program itself, depending upon the composition of the particular sequence, however, the minimum values may be adjusted and are set as indicated above.

In some embodiments, a functional equivalent, analogue or derivative of Ang-(1-7) is a fragment of the naturally-occurring Ang-(1-7). In some embodiments, a functional equivalent, analogue or derivative of Ang-(1-7) contains amino acid substitutions, deletions and/or insertions in the naturally-occurring Ang-(1-7). Ang-(1-7) functional equivalents, analogues or derivatives can be made by altering the amino acid sequences by substitutions, additions, and/or deletions. For example, one or more amino acid residues within the sequence of the naturally-occurring Ang-(1-7) (SEQ ID NO: 1) can be substituted by another amino acid of a similar polarity, which acts as a functional equivalent, resulting in a silent alteration. Substitution for an amino acid within the sequence may be selected from other members of the class to which the amino acid belongs. For example, the positively charged (basic) amino acids include arginine, lysine, and histidine. The nonpolar (hydrophobic) amino acids include leucine, isoleucine, alanine, phenylalanine, valine, proline, tryptophan, and methionine. The uncharged polar amino acids include serine, threonine, cysteine, tyrosine, asparagine, and glutamine. The negatively charged (acid) amino acids include glutamic acid and aspartic acid. The amino acid glycine may be included in either the nonpolar amino acid family or the uncharged (neutral) polar amino acid family. Substitutions made within a family of amino acids are generally understood to be conservative substitutions. For example, the amino acid sequence of a peptide inhibitor can be modified or substituted.

Examples of Ang-(1-7) functional equivalents, analogues and derivatives are described in the section entitled “Exemplary Angiotensin (1-7) Peptides” below.

An angiotensin-(1-7) peptide can be of any length. In some embodiments, an angiotensin-(1-7) peptide according to the present invention can contain, for example, from 4-25 amino acids (e.g., 4-20, 4-15, 4-14, 4-13, 4-12, 4-11, 4-10, 4-9, 4-8, 4-7 amino acids). In some embodiments, the linear peptide contains 4, 5, 6, 7, 8, 9, 10, 11, 12, 13, 14, 16, 17, 18, 19, 20, 21, 22, 23, 24, or 25 amino acids.

In some embodiments, an angiotensin-(1-7) peptide contains one or more modifications to increase protease resistance, serum stability and/or bioavailability. In some embodiments, suitable modifications are selected from pegylation, acetylation, glycosylation, biotinylation, substitution with D-amino acid and/or un-natural amino acid, and/or cyclization of the peptide.

As used herein, the term “amino acid,” in its broadest sense, refers to any compound and/or substance that can be incorporated into a polypeptide chain. In certain embodiments, an amino acid has the general structure H₂N—C(H)(R)—COOH. In certain embodiments, an amino acid is a naturally-occurring amino acid. In certain embodiments, an amino acid is a synthetic or un-natural amino acid (e.g., α,α-disubstituted amino acids, N-alkyl amino acids); in some embodiments, an amino acid is a d-amino acid; in certain embodiments, an amino acid is an 1-amino acid. “Standard amino acid” refers to any of the twenty standard amino acids commonly found in naturally occurring peptides including both 1- and d-amino acids which are both incorporated in peptides in nature. “Nonstandard” or “unconventional amino acid” refers to any amino acid, other than the standard amino acids, regardless of whether it is prepared synthetically or obtained from a natural source. As used herein, “synthetic or un-natural amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, and/or substitution with other chemical groups that can change the peptide's circulating half-life without adversely affecting its activity. Examples of unconventional or un natural amino acids include, but are not limited to, citrulline, omithine, norleucine, norvaline, 4-(E)-butenyl-4(R)-methyl-N-methylthreonine (MeBmt), N-methyl-leucine (MeLeu), aminoisobutyric acid, statine, and N-methyl-alanine (MeAla). Amino acids may participate in a disulfide bond. The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

In certain embodiments, angiotensin-(1-7) peptides contain one or more L-amino acids, D-amino acids, and/or un-natural amino acids.

In addition to peptides containing only naturally occurring amino acids, peptidomimetics or peptide analogs are also encompassed by the present invention. Peptide analogs are commonly used in the pharmaceutical industry as non-peptide drugs with properties analogous to those of the template peptide. The non-peptide compounds are termed “peptide mimetics” or peptidomimetics (Fauchere et al., Infect. Immun. 54:283-287 (1986); Evans et al., J Med. Chem. 30:1229-1239 (1987)). Peptide mimetics that are structurally related to therapeutically useful peptides and may be used to produce an equivalent or enhanced therapeutic or prophylactic effect. Generally, peptidomimetics are structurally similar to the paradigm polypeptide (i.e., a polypeptide that has a biological or pharmacological activity) such as naturally-occurring receptor-binding polypeptides, but have one or more peptide linkages optionally replaced by linkages such as —CH₂NH—, —CH₂S—, —CH₂—CH₂—, —CH═CH-(cis and trans), —CH₂SO—, —CH(OH)CH₂—, —COCH₂— etc., by methods well known in the art (Spatola, Peptide Backbone Modifications, Vega Data, 1(3):267 (1983); Spatola et al. Life Sci. 38:1243-1249 (1986); Hudson et al. Int. J Pept. Res. 14:177-185 (1979); and Weinstein. B., 1983, Chemistry and Biochemistry, of Amino Acids, Peptides and Proteins, Weinstein eds, Marcel Dekker, New-York). Such peptide mimetics may have significant advantages over naturally-occurring polypeptides including more economical production, greater chemical stability, enhanced pharmacological properties (e.g., half-life, absorption, potency, efficiency, etc.), reduced antigenicity and others.

Ang-(1-7) peptides also include other types of peptide derivatives containing additional chemical moieties not normally part of the peptide, provided that the derivative retains the desired functional activity of the peptide. Examples of such derivatives include (1)N-acyl derivatives of the amino terminal or of another free amino group, wherein the acyl group may be an alkanoyl group (e.g., acetyl, hexanoyl, octanoyl) an aroyl group (e.g., benzoyl) or a blocking group such as F-moc (fluorenylmethyl-0-CO—); (2) esters of the carboxy terminal or of another free carboxy or hydroxyl group; (3) amide of the carboxy-terminal or of another free carboxyl group produced by reaction with ammonia or with a suitable amine; (4) phosphorylated derivatives; (5) derivatives conjugated to an antibody or other biological ligand and other types of derivatives; and (6) derivatives conjugated to a polyethylene glycol (PEG) chain.

Ang-(1-7) peptides may be obtained by any method of peptide synthesis known to those skilled in the art, including synthetic (e.g., exclusive solid phase synthesis, partial solid phase synthesis, fragment condensation, classical solution synthesis, native-chemical ligation) and recombinant techniques. For example, the peptides or peptides derivatives can be obtained by solid phase peptide synthesis, which in brief, consist of coupling the carboxyl group of the C-terminal amino acid to a resin (e.g., benzhydrylamine resin, chloromethylated resin, hydroxymethyl resin) and successively adding N-alpha protected amino acids. The protecting groups may be any such groups known in the art. Before each new amino acid is added to the growing chain, the protecting group of the previous amino acid added to the chain is removed. Such solid phase synthesis has been disclosed, for example, by Merrifield, J Am. Chem. Soc. 85: 2149 (1964); Vale et al., Science 213:1394-1397 (1981), in U.S. Pat. Nos. 4,305,872 and 4,316,891, Bodonsky et al. Chem. Ind. (London), 38:1597 (1966); and Pietta and Marshall, Chem. Comm. 650 (1970) by techniques reviewed in Lubell et al. “Peptides” Science of Synthesis 21.11, Chemistry of Amides. Thieme, Stuttgart, 713-809 (2005). The coupling of amino acids to appropriate resins is also well known in the art and has been disclosed in U.S. Pat. No. 4,244,946. (Reviewed in Houver-Weyl, Methods of Organic Chemistry. Vol E22a. Synthesis of Peptides and Peptidomimetics, Murray Goodman, Editor-in-Chief, Thieme. Stuttgart. New York 2002).

Unless defined otherwise, the scientific and technological terms and nomenclature used herein have the same meaning as commonly understood by a person of ordinary skill to which this invention pertains. Generally, the procedures of cell cultures, infection, molecular biology methods and the like are common methods used in the art. Such standard techniques can be found in reference manuals such as, for example, Ausubel et al., Current Protocols in Molecular Biology, Wiley Interscience, New York, 2001; and Sambrook et al., Molecular Cloning: A Laboratory Manual, 3rd edition, Cold Spring Harbor Laboratory Press, N.Y., 2001.

During any process of the preparation of an Ang-(1-7) peptide, it may be desirable to protect sensitive reactive groups on any of the molecule concerned. This may be achieved by means of conventional protecting groups such as those described in Protective Groups In Organic Synthesis by T. W. Greene & P. G. M. Wuts, 1991, John Wiley and Sons, New-York; and Peptides: chemistry and Biology by Sewald and Jakubke, 2002, Wiley-VCR, Wheinheim p. 142. For example, alpha amino protecting groups include acyl type protecting groups (e.g., trifluoroacetyl, formyl, acetyl), aliphatic urethane protecting groups (e.g., t-butyloxycarbonyl (BOC), cyclohexyloxycarbonyl), aromatic urethane type protecting groups (e.g., fluorenyl-9-methoxy-carbonyl (Fmoc), benzyloxycarbonyl (Cbz), Cbz derivatives) and alkyl type protecting groups (e.g., triphenyl methyl, benzyl). The amino acids side chain protecting groups include benzyl (for Thr and Ser), Cbz (Tyr, Thr, Ser, Arg, Lys), methyl ethyl, cyclohexyl (Asp, His), Boe (Arg, His, Cys) etc. The protecting groups may be removed at a convenient subsequent stage using methods known in the art.

Further, Ang-(1-7) peptides may be synthesized according to the FMOC protocol in an organic phase with protective groups. Desirably, the peptides are purified with a yield of 70% with high-pressure liquid chromatography (HPLC) on a C 18 chromatography column and eluted with an acetonitrile gradient of 10-60%. The molecular weight of a peptide can be verified by mass spectrometry (reviewed in Fields, G. B. “Solid-Phase Peptide Synthesis” Methods in Enzymology. Vol. 289, Academic Press, 1997).

Alternatively, Ang-(1-7) peptides may be prepared in recombinant systems using, for example, polynucleotide sequences encoding the polypeptides. It is understood that a polypeptide may contain more than one of the above-described modifications within the same polypeptide.

While peptides may be effective in eliciting a biological activity in vitro, their effectiveness in vivo might be reduced by the presence of proteases. Serum proteases have specific substrate requirements. The substrate must have both L-amino acids and peptide bonds for cleavage. Furthermore, exopeptidases, which represent the most prominent component of the protease activity in serum, usually act on the first peptide bond of the peptide and require a free N-terminus (Powell et al., Pharm. Res. 10:1268-1273 (1993)). In light of this, it is often advantageous to use modified versions of peptides. The modified peptides retain the structural characteristics of the original L-amino acid peptides that confer the desired biological activity of Ang-(1-7) but are advantageously not readily susceptible to cleavage by protease and/or exopeptidases.

Systematic substitution of one or more amino acids of a consensus sequence with D-amino acid of the same type (e.g., D-lysine in place of L-lysine) may be used to generate more stable peptides. Thus, a peptide derivative or peptidomimetic of the present invention may be all L, all D or mixed D, L peptide, in either forward or reverse order. The presence of an N-terminal or C-terminal D-amino acid increases the in vivo stability of a peptide since peptidases cannot utilize a D-amino acid as a substrate (Powell et al., Pharm. Res. 10:1268-1273 (1993)). Reverse-D peptides are peptides containing D-amino acids, arranged in a reverse sequence relative to a peptide containing L-amino acids. Thus, the C-terminal residue of an L-amino acid peptide becomes N-terminal for the D-amino acid peptide, and so forth. Reverse D-peptides retain the same secondary conformation and therefore similar activity, as the L-amino acid peptides, but are more resistant to enzymatic degradation in vitro and in vivo, and thus can have greater therapeutic efficacy than the original peptide (Brady and Dodson, Nature 368:692-693 (1994); Jameson et al., Nature 368:744-746 (1994)). Similarly, a reverse-L peptide may be generated using standard methods where the C-terminus of the parent peptide becomes takes the place of the N-terminus of the reverse-L peptide. It is contemplated that reverse L-peptides of L-amino acid peptides that do not have significant secondary structure (e.g., short peptides) retain the same spacing and conformation of the side chains of the L-amino acid peptide and therefore often have the similar activity as the original L-amino acid peptide. Moreover, a reverse peptide may contain a combination of L- and D-amino acids. The spacing between amino acids and the conformation of the side chains may be retained resulting in similar activity as the original L-amino acid peptide.

Another effective approach to confer resistance to peptidases acting on the N-terminal or C-terminal residues of a peptide is to add chemical groups at the peptide termini, such that the modified peptide is no longer a substrate for the peptidase. One such chemical modification is glycosylation of the peptides at either or both termini. Certain chemical modifications, in particular N-terminal glycosylation, have been shown to increase the stability of peptides in human serum (Powell et al., Pharm. Res. 10:1268-1273 (1993)). Other chemical modifications which enhance serum stability include, but are not limited to, the addition of an N-terminal alkyl group, consisting of a lower alkyl of from one to twenty carbons, such as an acetyl group, and/or the addition of a C-terminal amide or substituted amide group. In particular, the present invention includes modified peptides consisting of peptides bearing an N-terminal acetyl group and/or a C-terminal amide group.

Substitution of non-naturally-occurring amino acids for natural amino acids in a subsequence of the peptides can also confer resistance to proteolysis. Such a substitution can, for instance, confer resistance to proteolysis by exopeptidases acting on the N-terminus without affecting biological activity. Examples of non-naturally-occurring amino acids include α,α-disubstituted amino acids, N-alkyl amino acids, C α-methyl amino acids, β-amino acids, and β-methyl amino acids. Amino acids analogs useful in the present invention may include, but are not limited to, β-alanine, norvaline, norleucine, 4-aminobutyric acid, orithine, hydroxyproline, sarcosine, citrulline, cysteic acid, cyclohexylalanine, 2-aminoisobutyric acid, 6-aminohexanoic acid, t-butylglycine, phenylglycine, o-phosphoserine, N-acetyl serine, N-formylmethionine, 3-methylhistidine and other unconventional amino acids. Furthermore, the synthesis of peptides with non-naturally-occurring amino acids is routine in the art.

In addition, constrained peptides comprising a consensus sequence or a substantially identical consensus sequence variation may be generated by methods well known in the art (Rizo and Gierasch, Ann. Rev. Biochem. 61:387-418 (1992)). For example, constrained peptides may be generated by adding cysteine residues capable of forming disulfide bridges and, thereby, resulting in a cyclic peptide. Cyclic peptides can be constructed to have no free N- or C-termini. Accordingly, they are not susceptible to proteolysis by exopeptidases, although they may be susceptible to endopeptidases, which do not cleave at peptide termini. The amino acid sequences of the peptides with N-terminal or C-terminal D-amino acids and of the cyclic peptides are usually identical to the sequences of the peptides to which they correspond, except for the presence of N-terminal or C-terminal D-amino acid residue, or their circular structure, respectively.

### Cyclic Peptides

In some embodiments, a functional equivalent, analogue or derivative of naturally-occurring Ang-(1-7) is a cyclic peptide. As used herein, a cyclic peptide has an intramolecular covalent bond between two non-adjacent residues. The intramolecular bond may be a backbone to backbone, side-chain to backbone or side-chain to side-chain bond (i.e., terminal functional groups of a linear peptide and/or side-chain functional groups of a terminal or interior residue may be linked to achieve cyclization). Typical intramolecular bonds include disulfide, amide and thioether bonds. A variety of means for cyclizing polypeptides are well known in the art, as are many other modifications that can be made to such peptides. For a general discussion, see International Patent Publication Nos. WO 01/53331 and WO 98/02452, the contents of which are incorporated herein by reference. Such cyclic bonds and other modifications can also be applied to the cyclic peptides and derivative compounds of this invention.

Cyclic peptides as described herein may comprise residues of L-amino acids, D-amino acids, or any combination thereof. Amino acids may be from natural or non-natural sources, provided that at least one amino group and at least one carboxyl group are present in the molecule; α- and β-amino acids are generally preferred. Cyclic peptides may also contain one or more rare amino acids (such as 4-hydroxyproline or hydroxylysine), organic acids or amides and/or derivatives of common amino acids, such as amino acids having the C-terminal carboxylate esterified (e.g., benzyl, methyl or ethyl ester) or amidated and/or having modifications of the N-terminal amino group (e.g., acetylation or alkoxycarbonylation), with or without any of a wide variety of side-chain modifications and/or substitutions (e.g., methylation, benzylation, t-butylation, tosylation, alkoxycarbonylation, and the like). Suitable derivatives include amino acids having an N-acetyl group (such that the amino group that represents the N-terminus of the linear peptide prior to cyclization is acetylated) and/or a C-terminal amide group (i.e., the carboxy terminus of the linear peptide prior to cyclization is amidated). Residues other than common amino acids that may be present with a cyclic peptide include, but are not limited to, penicillamine, β,β-tetramethylene cysteine, β,β-pentamethylene cysteine, β-mercaptopropionic acid, β-pentamethylene-mercaptopropionic acid, 2-mercaptobenzene, 2-mercaptoaniline, 2-mercaptoproline, ornithine, diaminobutyric acid, α-aminoadipic acid, m-aminomethylbenzoic acid and α,β-diaminopropionic acid.

Following synthesis of a linear peptide, with or without N-acetylation and/or C-amidation, cyclization may be achieved by any of a variety of techniques well known in the art. Within one embodiment, a bond may be generated between reactive amino acid side chains. For example, a disulfide bridge may be formed from a linear peptide comprising two thiol-containing residues by oxidizing the peptide using any of a variety of methods. Within one such method, air oxidation of thiols can generate disulfide linkages over a period of several days using either basic or neutral aqueous media. The peptide is used in high dilution to minimize aggregation and intermolecular side reactions. Alternatively, strong oxidizing agents such as h and K₃Fe(CN)₆ can be used to form disulfide linkages. Those of ordinary skill in the art will recognize that care must be taken not to oxidize the sensitive side chains of Met, Tyr, Trp or His. Within further embodiments, cyclization may be achieved by amide bond formation. For example, a peptide bond may be formed between terminal functional groups (i.e., the amino and carboxy termini of a linear peptide prior to cyclization). Within another such embodiment, the linear peptide comprises a D-amino acid. Alternatively, cyclization may be accomplished by linking one terminus and a residue side chain or using two side chains, with or without an N-terminal acetyl group and/or a C-terminal amide. Residues capable of forming a lactam bond include lysine, ornithine (Orn), α-amino adipic acid, m-aminomethylbenzoic acid, α,β-diaminopropionic acid, glutamate or aspartate. Methods for forming amide bonds are generally well known in the art. Within one such method, carbodiimide-mediated lactam formation can be accomplished by reaction of the carboxylic acid with DCC, DIC, ED AC or DCCI, resulting in the formation of an O-acylurea that can be reacted immediately with the free amino group to complete the cyclization. Alternatively, cyclization can be performed using the azide method, in which a reactive azide intermediate is generated from an alkyl ester via a hydrazide. Alternatively, cyclization can be accomplished using activated esters. The presence of electron withdrawing substituents on the alkoxy carbon of esters increases their susceptibility to aminolysis. The high reactivity of esters of p-nitrophenol, N-hydroxy compounds and polyhalogenated phenols has made these “active esters” useful in the synthesis of amide bonds. Within a further embodiment, a thioether linkage may be formed between the side chain of a thiol-containing residue and an appropriately derivatized α-amino acid. By way of example, a lysine side chain can be coupled to bromoacetic acid through the carbodiimide coupling method (DCC, EDAC) and then reacted with the side chain of any of the thiol containing residues mentioned above to form a thioether linkage. In order to form dithioethers, any two thiol containing side-chains can be reacted with dibromoethane and diisopropylamine in DMF.

### Exemplary Angiotensin-(1-7) Peptides

In certain aspects, the invention provides linear angiotensin-(1-7) peptides. As discussed above, the structure of naturally-occurring Ang-(1-7) is as follows:

(SEQ ID NO: 1)
Asp¹-Arg²-Val³-Tyr⁴-Ile⁵-His⁶-Pro⁷

The peptides and peptide analogs of the invention can be generally represented by the following sequence:

Xaa¹-Xaa²-Xaa³-Xaa⁴-Xaa⁵-Xaa⁶-Xaa⁷ (SEQ ID NO: 4), or a
pharmaceutically acceptable salt thereof.
Xaa¹ is any amino acid or a dicarboxylic acid. In certain embodiments, Xaa¹ is Asp, Glu, Asn, Acpc (1-aminocyclopentane carboxylic acid), Ala, Me₂Gly (N, N-dimethylglycine), Pro, Bet (betaine, 1-carboxy-N,N,N-trimethylmethanaminium hydroxide), Glu, Gly, Asp, Sar (sarcosine) or Suc (succinic acid). In certain such embodiments, Xaa¹ is a negatively-charged amino acid, such as Asp or Glu, typically Asp.
Xaa² is Arg, Lys, Ala, Cit (citrulline), Orn (omithine), acetylated Ser, Sar, D-Arg and D-Lys. In certain embodiments, Xaa2 is a positively-charged amino acid such as Arg or Lys, typically Arg.
Xaa³ is Val, Ala, Leu, Nle (norleucine), Ile, Gly, Lys, Pro, HydroxyPro (hydroxyproline), Aib (2-aminoisobutyric acid), Acpc or Tyr. In certain embodiments, Xaa³ is an aliphatic amino acid such as Val, Leu, Ile or Nle, typically Val or Nle.
Xaa⁴ is Tyr, Tyr(PO₃), Thr, Ser, homoSer (homoserine), azaTyr (aza-α¹-homo-L-tyrosine) or Ala. In certain embodiments, Xaa⁴ is a hydroxyl-substituted amino acid such as Tyr, Ser or Thr, typically Tyr.
Xaa⁵ is Ile, Ala, Leu, norLeu, Val or Gly. In certain embodiments, Xaa⁵ is an aliphatic amino acid such as Val, Leu, Ile or Nle, typically Ile.
Xaa⁶ is His, Arg or 6-NH₂-Phe (6-aminophenylalaine). In certain embodiments, Xaa⁶ is a fully or partially positively-charged amino acid such as Arg or His.

### Xaa⁷ is Cys, Pro or Ala.

In certain embodiments, one or more of Xaa¹-Xaa⁷ is identical to the corresponding amino acid in naturally-occurring Ang-(1-7). In certain such embodiments, all but one or two of Xaa¹-Xaa⁷ are identical to the corresponding amino acid in naturally-occurring Ang-(1-7). In other embodiments, all of Xaa¹-Xaa⁶ are identical to the corresponding amino acid in naturally-occurring Ang-(1-7).

In certain embodiments, Xaa³ is Nle. When Xaa³ is Nle, one or more of Xaa¹-Xaa² and Xaa⁴⁻⁷ are optionally identical to the corresponding amino acid in naturally-occurring Ang-(1-7). In certain such embodiments, all but one or two of Xaa¹-Xaa² and Xaa⁴⁻⁷ are identical to the corresponding amino acid in naturally-occurring Ang-(1-7). In other embodiments, all of Xaa¹-Xaa² and Xaa⁴⁻⁷ are identical to the corresponding amino acid in naturally-occurring Ang-(1-7), resulting in the amino acid sequence: Asp¹-Arg²-Nle³-Tyr⁴-Ile⁵-His⁶-Pro⁷ (SEQ ID NO: 5).

In certain embodiments, the peptide has the amino acid sequence Asp¹-Arg²-Val³-Ser⁴-Ile⁵-His⁶-Cys⁷ (SEQ ID NO: 6) or Asp¹-Arg²-Val³-ser^{o}-Ile⁵-His⁶-Cys⁷ (SEQ ID NO: 2).

### Exemplary Cyclic Angiotensin (1-7) Peptides

In certain aspects, the invention provides a cyclic angiotensin-(1-7) (Ang-(1-7)) peptide analog comprising a linkage, such as between the side chains of amino acids corresponding to positions Tyr⁴ and Pro⁷ in Ang. These peptide analogs typically comprise 7 amino acid residues, but can also include a cleavable sequence. As discussed in greater detail below, the invention includes fragments and analogs where one or more amino acids are substituted by another amino acid (including fragments). One example of such an analog is Asp¹-Arg²-Val³-Ser⁴-Ile⁵-His⁶-Cys⁷ (SEQ ID NO: 22), wherein a linkage is formed between Ser⁴ and Cys⁷.

Although the following section describes aspects of the invention in terms of a thioether bond linking residues at the 4- and 7-positions, it should be understood that other linkages (as described above) could replace the thioether bridge and that other residues could be cyclized. A thioether bridge is also referred to as a monosulfide bridge or, in the case of Ala-S-Ala, as a lanthionine bridge. Thioether bridge-containing peptides can be formed by two amino acids having one of the following formulas:

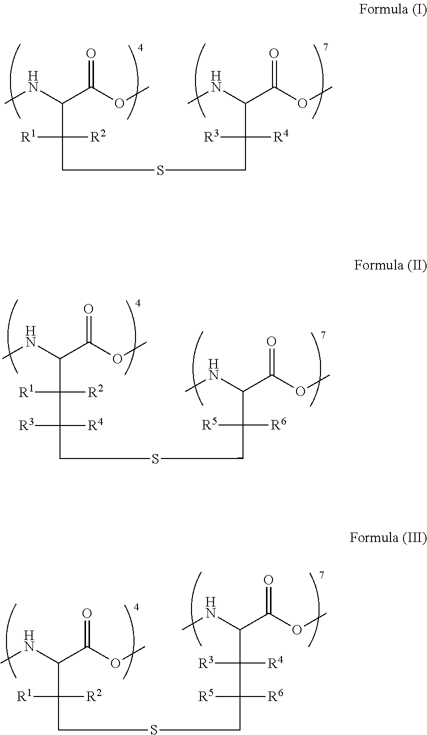

In these formulae, R¹, R², R³, R⁴, R⁵ and R⁶ are independently H, an alkyl (e.g., C1-C6 alkyl, C1-C4 alkyl) or an aralkyl group, where the alkyl and aralkyl groups are optionally substituted with one or more halogen, —OH or —NRR′ groups (where R and R′ are independently —H or C1-C4 alkyl). In certain embodiments, R1 R2, R3, R4, R5 and R6 are each independently —H or —CH3, such where all are —H.

In certain embodiments, the invention provides an Ang analog or derivative comprising a thioether bridge according to formula (I). Typically, R¹, R², R³ and R⁴ are independently selected from —H and —CH₃. Peptides comprising a thioether bridge according to formula (I) can be produced, for example, by lantibiotic enzymes or by sulfur extrusion of a disulfide. In one example, the disulfide from which the sulfur is extruded can be formed by D-cysteine in position 4 and L-cysteine in position 7 or by D-cysteine in position 4 and L-penicillamine in position 7 (see, e.g., Galande, Trent and Spatola (2003) Biopolymers 71, 534-551).

In other embodiments, the linkage of the two amino acids can be the bridges depicted in Formula (II) or Formula (III). Peptides comprising a thioether bridge according to Formula (II) can be made, for example, by sulfur extrusion of a disulfide formed by D-homocysteine in position 4 and L-cysteine in position 7. Similarly, peptides comprising a thioether bridge as in Formula (III) can be made, for example, by sulfur extrusion of a disulfide formed by D-cysteine in position 4 and L-homocysteine in position 7.

As discussed above, the Ang analogs and derivatives of the invention vary in length and amino acid composition. The Ang analogs and derivatives of the invention preferably have biological activity or are an inactive precursor molecule that can be proteolytically activated (such as how angiotensin(I), with 10 amino acids, is converted to active fragments by cleavage of 2 amino acids). The size of an Ang analog or derivative can vary but is typically between from about 5 to 10 amino acids, as long as the “core” pentameric segment comprising the 3-7 Nle-thioether-ring structure is encompassed. The amino acid sequence of an analog or derivative of the invention can vary, typically provided that it is biologically active or can become proteolytically activated. Biological activity of an analog or derivative can be determined using methods known in the art, including radioligand binding studies, in vitro cell activation assays and in vivo experiments. See, for example, Godeny and Sayeski, (2006)Am. J Physiol. Cell. Physiol. 291: C1297-1307; Sarr et al., Cardiovasc. Res. (2006) 71:794 802; and Koziarz et al., (1933) Gen. Pharmacol. 24:705-713.

Ang analogs and derivatives where only the length of the peptide is varied include the following:

a 4,7-cyclized analog designated [Cyc^{4 7}]Ang-(1-7), which is derived from natural Ang-(1-7) (Asp¹-Arg²-Val³-Cyc⁴-Ile⁵-His⁶-Cyc⁷, SEQ ID NO:7).
a 4,7-cyclized analog designated [Nle³, Cyc4-7]Ang-(1-10), which is derived from natural
Angiotensin I (Ang-(1-10)) (Asp¹-Arg²-Nle³-Cyc⁴-Ile⁵-His⁶-Cyc⁷-Phe⁸-His⁹-Leu¹⁰ SEQ ID N0:8);
a 4,7-cyclized analog designated [Nle³, Cyc4-7]Ang-(1-8), which is derived from natural Angiotensin II (Ang-(1-8)) (Asp¹-Arg²-Nle³-Cyc⁴-Ile⁵-His⁶-Cyc⁷-Phe⁸, SEQ ID NO:9);
a 4,7-cyclised analog designated [Nle³, Cyc^{4 7}]Ang-(2-8), which is derived from natural Angiotensin III (Ang-(2-8)) (Arg²-Nle³-Cyc⁴-Ile⁵-His⁶-Cyc⁷-Phe⁸, SEQ ID NO: 10);
a 4,7-cyclised analog designated [Nle³, Cyc^{4 7}]Ang-(3-8), which is derived from natural Angiotensin IV (Ang-(3-8)) (Nle³-Cyc⁴-Ile⁵-His⁶-Cyc⁷-Phe⁸, SEQ ID NO: 11);
a 4,7-cyclised analog designated [Nle³, Cyc^{4 7}]Ang-(1-7) derived from natural Ang-(1-7) (Asp¹-Arg²-Nle³-Cyc⁴-Ile⁵-His⁶-Cyc⁷, SEQ ID NO:12); and
a 4,7-cyclised analog designated [Nle³, Cyc^{4 7}]Ang-(1-9) derived from natural Ang-(1-9) (Asp¹-Arg²-Nle³-Cyc⁴-Ile⁵-His⁶-Cyc⁷-Phe⁸-His⁹, SEQ ID NO: 13).
These analogs can have one of the thioether bridges shown in Formulae (I)-(III) as the Cyc^{4 7} moiety, for example, where Cyc⁴ and Cyc⁷ are represented by Formula (I), such as where R¹-R⁴ are each —H or —CH3, typically —H.

As compared to the amino acid sequence of the natural angiotensin peptide, the amino acids at positions 4 and 7 of the Cyc4-7-analog are modified to allow introduction of the thioether-ring structures shown above. In addition to the length of the Ang analogs, the amino acids at positions other than 3, 4 and 7 can be the same or different from the naturally-occurring peptide, typically provided that the analog retains a biological function. For analogs of inactive precursors, like [Cyc4-7]Ang-(1-10), biological function refers to one or both of an analog's susceptibility to angiotensin-converting enzymes that can cleave it to a biologically active fragment (e.g. Ang-(1-8) or Ang-(1-7)) or the biological activity of the fragment itself. In certain embodiments, an Ang analog or derivative of the invention has no intrinsic function but inhibits the effects of one or more naturally-occurring angiotensin compounds.

In certain embodiments, an Ang analog of the invention is represented by Formula (IV):

(IV, SEQ ID NO: 14)
Xaa¹-Xaa²-Xaa³-Cyc⁴-Xaa⁵-Xaa⁶-Cyc⁷

Xaa¹ is any amino acid, but typically a negatively-charged amino acid such as Glu or Asp, more typically Asp.
Xaa² is a positively-charged amino acid such as Arg or Lys, typically Arg.
Xaa³ is an aliphatic amino acid, such as Leu, Ile or Val, typically Val.
Cyc⁴ forms a thioether bridge in conjunction with Cyc⁷. Cyc⁴ can be a D-stereoisomer and/or a L-stereoisomer, typically a D-stereoisomer. Examples of Cyc⁴ (taken with Cyc⁷) are shown in Formulas (I), (II) and (III). Typically, the R groups in Formulae (I), (II) and (III) are —H or —CH3, especially —H.
Xaa⁵ is an aliphatic amino acid, such as Leu, Ile or Val, typically Ile.

### Xaa⁶ is His.

Cyc⁷ forms a thioether bridge in conjunction with Cyc⁴, such as in Formula (I), (II) or (III). Cyc⁷ can be a D-stereoisomer and/or a L-stereoisomer, typically a L-stereoisomer. Examples of Cyc⁷ (taken with Cyc⁴) are shown in Formulas (I), (II), (III) and (IV). Typically, the R groups in Formulae (I), (II)) and (III) and (IV) are —H or —CH3, especially —H.

In certain embodiments, one or more of Xaa¹-Xaa⁶ (excluding Cyc⁴ and Cyc⁷) is identical to the corresponding amino acid in naturally-occurring Ang-(1-7). In certain such embodiments, all but one or two of Xaa¹-Xaa⁶ are identical to the corresponding amino acid in naturally-occurring Ang-(1-7). In other embodiments, all of Xaa¹-Xaa⁶ are identical to the corresponding amino acid in naturally-occurring Ang-(1-7).

In certain embodiments, Cyc⁴ and Cyc⁷ are independently selected from Abu (2-aminobutyric acid) and Ala (alanine), where Ala is present in at least one position. Thus, cyclic analogs can have a thioether linkage formed by -Ala⁴-S-Ala⁷-(Formula (I), where R¹—R⁴ are each —H); -Ala⁴-S-Abu⁷-(Formula (I): R¹—R³ are —H and R⁴ is —CH₃) or-Abu⁴-S-Ala⁷-(Formula (I): R¹, R³ and R⁴ are —H and R² is —CH₃). Specific examples of cyclic analogs comprise a-Abu⁴-S— Ala⁷- or—Ala⁴-S— Ala⁷-linkage.

In certain embodiments, the invention provides an Ang-(1-7) analog with a thioether-bridge between position 4 and position 7 having the amino acid sequence Asp¹-Arg²-Val³-Abu⁴-Ile⁵-His⁶-Ala⁷ (SEQ ID NO: 15) or the amino acid sequence Asp¹-Arg²-Val³-Ala⁴-Ile⁵-His⁶-Ala⁷ (SEQ ID NO: 16), which are represented by the following structural diagrams:

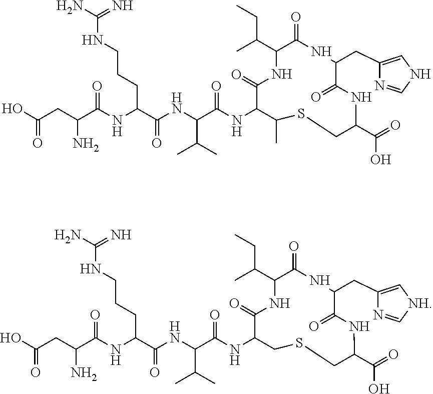 [SMILES: CCC(C)C1NC(=O)C(NC(=O)C(NC(=O)C(CCCNC(=N)N)NC(=O)C(N)CC(=O)O)C(C)C)C(C)SCC(C(=O)O)NC(=O)C(Cc2c[nH]cn2)NC1=O ; CCC(C)C1NC(=O)C(NC(=O)C(NC(=O)C(CCCNC(=N)N)NC(=O)C(N)CC(=O)O)C(C)C)CSCC(C(=O)O)NC(=O)C(Cc2c[nH]cn2)NC1=O]

In certain embodiments, an Ang analog or derivative of the invention is represented by Formula (V):

(V, SEQ ID NO: 17)
Xaa¹-Xaa²-Nle³-Cyc⁴-Xaa⁵-Xaa⁶-Cyc⁷-Xaa⁸-Xaa⁹-Xaa¹⁰

As discussed above, one or more of Xaa¹, Xaa², Xaa⁸, Xaa⁹ and Xaa¹⁰ are absent in certain embodiments.

For example,
Xaa¹⁰ is absent, (2) Xaa⁹ and Xaa¹⁰ are absent, (3) Xaa⁸, Xaa⁹ and Xaa¹⁰ are absent, (4) Xaa¹ is absent, (5) Xaa¹ and Xaa¹⁰ are absent, (6) Xaa⁸, Xaa⁹ and Xaa¹⁰ are absent, (7) Xaa¹, Xaa⁸, Xaa⁹ and Xaa¹⁰ are absent, (8) Xaa¹ and Xaa² are absent, (9) Xaa¹, Xaa² and Xaa¹⁰ are absent, (10) Xaa¹, Xaa², Xaa⁹ and Xaa¹⁰ are absent, or (11) Xaa¹, Xaa², Xaa⁸, Xaa⁹ and Xaa¹⁰ are absent. For each of these embodiments, the remaining amino acids have the values described below.
Xaa¹, when present, is any amino acid, but typically a negatively charged amino acid such as Glu or Asp, more typically Asp.
Xaa², when present, is a positively, charged amino acid such as Arg or Lys, typically Arg.
Nle³ is norleucine.
Cyc⁴ forms a thioether bridge in conjunction with Cyc⁷.Cyc⁴ can be a D-stereoisomer and/or a L-stereoisomer, typically a D-stereoisomer. Examples of Cyc⁴ (taken with Cyc⁷) are shown in Formulas (I), (II) and (III). Typically, the R groups in Formulae (I), (II) and (III) are —H or —CH, especially —H.
Xaa⁵ is an aliphatic amino acid, such as Leu, Nle, Ile or Val, typically Ile.

### Xaa⁶ is His.

Cyc⁷ forms a thioether bridge in conjunction with Cyc⁴, such as in Formula (I), (II) or (III).
Cyc⁷ can be a D-stereoisomer and/or a L-stereoisomer, typically a L-stereoisomer. Examples of Cyc7 (taken with Cyc) are shown in Formulas (I), (II) and (III). Typically, the R groups in Formulae (I), (II) and (III) are —H or —CH, especially —H.
Xaa⁸, when present, is any amino acid other than Pro, typically Phe or Ile. In certain embodiments, Ile results in an inhibitor of Ang (1-8). In certain embodiments, Phe maintains the biological activity of Ang (1-8) or Ang (1-10).
Xaa⁹, when present, is His.
Xaa¹⁰ when present, is an aliphatic residue, for example, Ile, Val or Leu, typically Leu.

In certain embodiments, one or more of Xaa¹-Xaa¹⁰ (excluding Nle³, Cyc⁴ and Cyc⁷) is identical to the corresponding amino acid in naturally-occurring Ang (including Ang-(1-7), Ang (1-8), Ang (1-9), Ang (1-10), Ang (2-7), Ang(2-8), Ang (2-9), Ang (2-10), Ang (3-8), Ang (3-9) and Ang(3-10). In certain such embodiments, all but one or two of Xaa¹-Xaa¹⁰ (for those present) are identical to the corresponding amino acid in naturally-occurring Ang. In other embodiments, all of Xaa¹-Xaa¹⁰ (for those present) are identical to the corresponding amino acid in naturally-occurring Ang.

In certain embodiments, Cyc4 and Cyc7 are independently selected from Abu (2-aminobutyric acid) and Ala (alanine), where Ala is present at least one position. Thus, encompassed are cyclic analogs comprising a thioether linkage formed by Ala⁴-S-Ala⁷-(Formula (I), where R¹—R⁴ are each —H); -Ala⁴-S-Abu⁷-(Formula (I): R¹—R³ are H and R⁴ is —CH₃) or-Abu⁴-S-Ala⁷-(Formula (I): R¹—R³ and R⁴ are —H and R² is CH₃). Specific cyclic analogs comprise a-Abu⁴-S-Ala⁷- or Ala⁴-S-Ala⁷-linkage.

In particular, the invention provides an Ang-(1-7) analog or derivative with a thioether-bridge between position 4 and position 7 having the amino acid sequence Asp¹-Arg²-Nle³-Abu⁴-Ile⁵-His⁶-Ala⁷ (SEQ ID NO:18) or the amino acid sequence Asp¹-Arg²-Nle³-Ala⁴-Ile⁵-His⁶-Ala⁷ (SEQ ID NO:19). In another aspect, the invention provides an Ang-(1-8) analog or derivative with a thioether-bridge between position 4 and position 7 having Ang-(1-8) antagonistic activity, in particular an Ang(1-8) analog or derivative having the amino acid sequence Asp¹-Arg²-Nle³-Abu⁴-Ile⁵-His⁶-Ala⁷-Ile⁸ (SEQ ID NO:20), or the amino acid sequence Asp¹-Arg²-Nle³-Ala⁴-Ile⁵-His⁶-Ala⁷-Ile⁸ (SEQID NO:21).

An alkyl group is a straight chained or branched nonaromatic hydrocarbon that is completely saturated. Typically, a straight chained or branched alkyl group has from 1 to about 20 carbon atoms, preferably from 1 to about 10. Examples of straight chained and branched alkyl groups include methyl, ethyl, n-propyl, iso-propyl. n-butyl, sec-butyl, tert-butyl, pentyl, hexyl, pentyl and octyl. A C1-C4 straight chained or branched alkyl group is also referred to as a “lower alkyl” group.

An aralkyl group is an alkyl group substituted by an aryl group. Aromatic (aryl) groups include carbocyclic aromatic groups such as phenyl, naphthyl, and anthracyl, and heteroaryl groups such as imidazolyl, thienyl, furyl, pyridyl, pyrimidyl, pyranyl, pyrazolyl pyrrolyl pyrazinyl, thiazolyl, oxazolyl, and tetrazolyl. Aromatic groups also include fused polycyclic aromatic ring systems in which a carbocyclic aromatic ring or heteroaryl ring is fused to one or more other heteroaryl rings. Examples include benzothienyl, benzofuryl, indolyl, quinolinyl, benzothiazole, benzoxazole, benzimidazole, quinolinyl, isoquinolinyl and isoindolyl.

### Ang-(1-7) Receptor Agonists

In some embodiments, the present invention provides methods of treating lung disease including administering to a subject who is suffering from or susceptible to one or more respiratory complications an angiotensin (1-7) receptor agonist. As used herein, the term “angiotensin-(1-7) receptor agonist” encompasses any molecule that has a positive impact in a function of an angiotensin-(1-7) receptor, in particular, the G-protein coupled Mas receptor. In some embodiments, an angiotensin-(1-7) receptor agonist directly or indirectly enhances, strengthens, activates and/or increases an angiotensin-(1-7) receptor (i.e., the Mas receptor) activity. In some embodiments, an angiotensin-(1-7) receptor agonist directly interacts with an angiotensin-(1-7) receptor (i.e., the Mas receptor). Such agonists can be peptidic or non-peptidic including, e.g., proteins, chemical compounds, Small molecules, nucleic acids, antibodies, drugs, ligands, or other agents. In some embodiments, the angiotensin (1-7) receptor agonist is a non-peptidic agonist.

An exemplary class of angiotensin-(1-7) receptor agonists are 1-(p-thienylbenzyl) imidazoles. Examples of these non peptide angiotensin-(1-7) receptor agonists are represented by Structural Formula (VI):

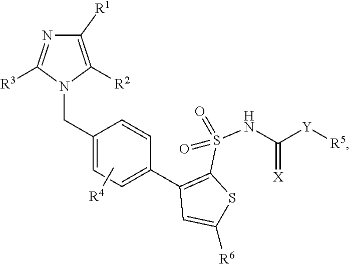

or pharmaceutically acceptable salts thereof, wherein:
R¹ is halogen, hydroxyl, (C₁-C₄)-alkoxy, (C₁-C₅)-alkoxy wherein 1 to 6 carbon atoms are replaced by the heteroatoms O, S, or NH (preferably by O), (C₁-C₄)-alkoxy substituted by a saturated cyclic ether such as tetrahydropyran or tetrahydrofuran, O—(C₁-C₄)-alkenyl, O (C₁-C₄)-alkylaryl, or aryloxy that is unsubstituted or substituted by a substituent selected from halogen, (C—C)-alkyl, (C—C)-alkoxy and trifluoromethyl;
R² is CHO, COOH, or (3)CO—O (C₁-C₄)-alkyl:
R³ is (C₁-C₄)-alkyl or aryl;
R⁴ is hydrogen, halogen (chloro, bromo, fluoro), or (C₁-C₄)-alkyl;
X is oxygen or Sulfur,
Y is oxygen or —NH—;
R⁵ is hydrogen, (C₁-C₆)-alkyl; or (C₁-C₄)-alkylaryl, where R⁵ is hydrogen when Y is NH—; and
R⁶ is (C¹-C⁵)-alkyl.
In certain embodiments, R¹ is not halogen when R is COOH or CO—O—(C—C)-alkyl.

In some embodiments, an angiotensin-(1-7) receptor agonist is AVE 0991, 5-formyl-4-methoxy-2-phenyl-14-2-(ethylaminocarbonylsulfonamido)-5-isobutyl-3-thienyl phenyl-methyl-imidazole, which is represented by the following structure:

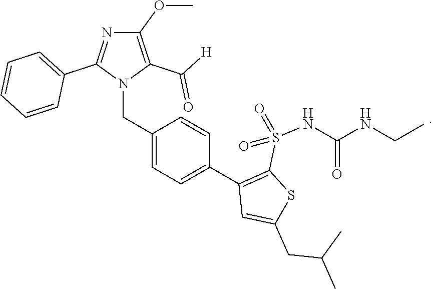 [SMILES: CCNC(=O)NS(=O)(=O)c1sc(CC(C)C)cc1-c1ccc(Cn2c(-c3ccccc3)nc(OC)c2C=O)cc1]

Another exemplary class of angiotensin-(1-7) receptor agonists are p-thienylbenzylamides. Examples of these non-peptide angiotensin-(1-7) receptor agonists are represented by Structural Formula (VII):

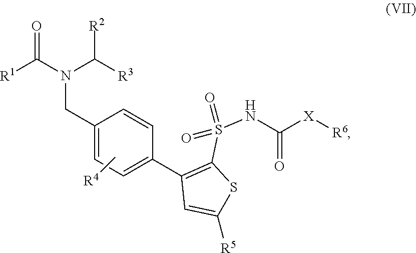

or a pharmaceutically acceptable salt thereof, wherein:
R¹ is (C₁-C₅)-alkyl that is unsubstituted or substituted by a radical chosen from NH₂, halogen, O—(C₁-C₃)-alkyl, CO—O—(C₁-C₃)-alkyl and CO₂H, (C₃-C₈)-cycloalkyl, (C₁-C₃)-alkyl-(C₃-C₈)-cycloalkyl, (C₆-C₁₀)-aryl that is unsubstituted or substituted by a radical chosen from halogen and O—(C₁-C₃)-alkyl, (C₁-C₃)-alkyl-(C₆-C₁₀)-aryl where the aryl radical is unsubstituted or substituted by a radical chosen from halogen and O—(C₁-C₃)-alkyl, (C₁-C₅)-heteroaryl, or (C₁-C₃)-alkyl-(C₁-C₅)-heteroaryl;
R² is hydrogen, (C₁-C₆)-alkyl that is unsubstituted or substituted by a radical chosen from halogen and O—(C₁-C₃)-alkyl, (C₃-C₈)-cycloalkyl, (C₁-C₃)-alkyl-(C₃-C₈)-cycloalkyl, (C₆-C₁₀)-aryl that is unsubstituted or substituted by a radical chosen from among halogen, O—(C₁-C₃)-alkyl and CO—O—(C₁-C₃)-alkyl, or (C₁-C₃)-alkyl-(C₆-C₁₀)-aryl that is unsubstituted or substituted by a radical chosen from halogen and O—(C₁-C₃)-alkyl;
R³ is hydrogen, COOH, or COO—(C₁-C₄)-alkyl;
R⁴ is hydrogen, halogen; or (C₁-C₄)-alkyl;
R⁵ is hydrogen or (C₁-C₆)-alkyl;
R⁶ is hydrogen, (C₁-C₆)-alkyl, (C₁-C₃)-alkyl-(C₃-C₈)-cycloalkyl, or (C₂-C₆)-alkenyl; and
X is oxygen or NH.

Additional examples of angiotensin-(1-7) receptor agonists are described in U.S. Pat. No. 6,235,766, the contents of which are incorporated by reference herein.

Various angiotensin-(1-7) receptor agonists described above can be present as pharmaceutically acceptable salts. As used herein, “a pharmaceutically acceptable salt” refers to salts that retain the desired activity of the peptide or equivalent compound, but preferably do not detrimentally affect the activity of the peptide or other component of a system, which uses the peptide. Examples of such salts are acid addition salts formed with inorganic acids, for example, hydrochloric acid, hydrobromic acid, sulfuric acid, phosphoric acid, nitric acid, and the like.

Salts may also be formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, pamoic acid, alginic acid, polyglutamic acid, and the like. Salts formed from a cationic material may utilize the conjugate base of these inorganic and organic acids. Salts may also be formed with polyvalent metal cations such as zinc, calcium, bismuth, barium, magnesium, aluminum, copper, cobalt, nickel and the like or with an organic cation formed from N,N′-dibenzylethylenediamine or ethylenediamine, or combinations thereof (e.g., a zinc tannate salt). The non-toxic, physiologically acceptable salts are preferred.

The salts can be formed by conventional means such as by reacting the free acid or free base forms of the product with one or more equivalents of the appropriate acid or base in a solvent or medium in which the salt is insoluble, or in a solvent such as water which is then removed in vacuo or by freeze-drying, or by exchanging the cations of an existing salt for another cation on a suitable ion exchange resin.

An alkyl group is a straight chained or branched non-aromatic hydrocarbon that is completely saturated. Typically, a straight chained or branched alkyl group has from 1 to about 20 carbon atoms, preferably from 1 to about 10. Examples of straight chained and branched alkyl groups include methyl, ethyl, n-propyl, iso-propyl, n-butyl, sec-butyl, tert-butyl, pentyl, hexyl, pentyl and octyl. A C 1-C4 straight chained or branched alkyl group is also referred to as a “lower alkyl” group.

An alkenyl group is a straight chained or branched non-aromatic hydrocarbon that is includes one or more double bonds. Typically, a straight chained or branched alkenyl group has from 2 to about 20 carbon atoms, preferably from 2 to about 10. Examples of straight chained and branched alkenyl groups include ethenyl, n-propenyl, and n-butenyl.

Aromatic (aryl) groups include carbocyclic aromatic groups such as phenyl, naphthyl, and anthracyl, and heteroaryl groups such as imidazolyl, thienyl, furyl, pyridyl, pyrimidyl, pyranyl, pyrazolyl, pyrrolyl, pyrazinyl, thiazolyl, oxazolyl, and tetrazolyl. Aromatic groups also include fused polycyclic aromatic ring systems in which a carbocyclic aromatic ring or heteroaryl ring is fused to one or more other heteroaryl rings. Examples include benzothienyl, benzofuryl, indolyl, quinolinyl, benzothiazole, benzoxazole, benzimidazole, quinolinyl, isoquinolinyl and isoindolyl.

An aralkyl group is an alkyl group substituted by an aryl group.

### Formulations

In accordance with the methods of the invention, an Ang-(1-7) peptide or angiotensin (1-7) receptor agonist as described herein of the invention can be administered to a subject alone (e.g., as a purified peptide or compound), or as a component of a composition or medicament (e.g., in the manufacture of a medicament for the treatment of the disease), as described herein. The compositions can be formulated with a physiologically acceptable carrier or excipient to prepare a pharmaceutical composition. The carrier and composition can be sterile. The formulation should suit the mode of administration, for example intravenous, subcutaneous or oral administration. Methods of formulating compositions are known in the art (see, e.g., Remington's Pharmaceuticals Sciences, 17th Edition, Mack Publishing Co., (Alfonso R. Gennaro, editor) (1989)).

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents (e.g., lubricants, preservatives, stabilizers, wetting agents, emulsifiers, salts for influencing osmotic pressure, buffers, coloring and/or aromatic substances and the like) which do not deleteriously react with the active compounds or interference with their activity. In a preferred embodiment, a water-soluble carrier suitable for intravenous administration is used. In some embodiments, Ang (1-7) is formulated with cyclodextrin for oral administration as described in, for example, WO 94/20136, the contents of which are incorporated herein by reference.

The composition or medicament, if desired, can also contain minor amounts of wetting or emulsifying agents, or pH buffering agents. The composition can be a liquid solution, suspension, emulsion, sustained release formulation, or powder. The composition can also be formulated as a suppository, with traditional binders and carriers such as triglycerides.

The composition or medicament can be formulated in accordance with the routine procedures as a pharmaceutical composition adapted for administration to human beings. For example, in a preferred embodiment, a composition for intravenous administration typically is a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

An Ang-(1-7) peptide or angiotensin (1-7) receptor agonist as described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

An Ang-(1-7) peptide or angiotensin (1-7) receptor agonist as described herein (or a composition or medicament containing an Ang-(1-7) peptide or angiotensin (1-7) receptor agonist described herein) is administered by any appropriate route. In some embodiments, an Ang-(1-7) peptide or angiotensin (1-7) receptor agonist described herein is administered subcutaneously. As used herein, the term “subcutaneous tissue”, is defined as a layer of loose, irregular connective tissue immediately beneath the skin. For example, the subcutaneous administration may be performed by injecting a composition into areas including, but not limited to, thigh region, abdominal region, gluteal region, or scapular region. In some embodiments, an Ang-(1-7) peptide or angiotensin (1-7) receptor agonist described herein is administered intravenously. Alternatively, an Ang-(1-7) peptide or angiotensin (1-7) receptor agonist described herein (or a composition or medicament containing an Ang-(1-7) peptide or angiotensin (1-7) receptor agonist described herein) can be administered by inhalation, parenterally, intradermally, transdermally, rectally, or transmucosally. More than one route can be used concurrently, if desired.

In some embodiments, a composition is administered in a therapeutically effective amount and/or according to a dosing regimen that is correlated with a particular desired outcome (e.g., with treating or reducing severity of lung disease).

Particular doses or amounts to be administered in accordance with the present invention may vary, for example, depending on the nature and/or extent of the desired outcome, on particulars of route and/or timing of administration, and/or on one or more characteristics (e.g., weight, age, personal history, genetic characteristic, lifestyle parameter, severity of pulmonary defect and/or level of risk of pulmonary defect, etc., or combinations thereof). Such doses or amounts can be determined by those of ordinary skill. In some embodiments, an appropriate dose or amount is determined in accordance with standard clinical techniques. For example, in some embodiments, an appropriate dose or amount is a dose or amount sufficient to reduce a disease severity index score by 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100% or more. For example, in some embodiments, an appropriate dose or amount is a dose or amount sufficient to reduce a disease severity index score by 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100%. Alternatively or additionally, in some embodiments, an appropriate dose or amount is determined through use of one or more in vitro or in vivo assays to help identify desirable or optimal dosage ranges or amounts to be administered.

In various embodiments, an Ang-(1-7) peptide or angiotensin (1-7) receptor agonist is administered at a therapeutically effective amount. As used herein, the term “therapeutically effective amount” is largely determined based on the total amount of the therapeutic agent contained in the pharmaceutical compositions of the present invention. Generally, a therapeutically effective amount is sufficient to achieve a meaningful benefit to the subject (e.g., treating, modulating, curing, preventing and/or ameliorating the underlying disease or condition). In some particular embodiments, appropriate doses or amounts to be administered may be extrapolated from dose-response curves derived from in vitro or animal model test systems.

Therapeutically effective dosage amounts of angiotensin (1-7) peptides or angiotensin (1-7) receptor agonists, including derivatives, analogs, and/or salts may be present in varying amounts in various embodiments. For example, in some embodiments, a therapeutically effective amount of an angiotensin (1-7) peptide may be an amount ranging from about 10-1000 mg (e.g., about 20 mg-1,000 mg, 30 mg 1,000 mg, 40 mg-1,000 mg, 50 mg-1,000 mg, 60 mg-1,000 mg, 70 mg-1,000 mg, 80 mg-1,000 mg, 90 mg-1,000 mg, about 10-900 mg, 10-800 mg, 10-700 mg, 10-600 mg, 10-500 mg, 100-1000 mg, 100-900 mg, 100-800 mg, 100-700 mg, 100-600 mg, 100-500 mg, 100-400 mg, 100-300 mg, 200-1000 mg, 200-900 mg, 200-800 mg, 200-700 mg, 200-600 mg, 200-500 mg, 200-400 mg, 300-1000 mg, 300-900 mg, 300-800 mg, 300-700 mg, 300-600 mg, 300-500 mg, 400 mg-1,000 mg, 500 mg-1,000 mg, 100 mg-900 mg, 200 mg-800 mg, 300 mg-700 mg, 400 mg-700 mg, and 500 mg 600 mg). In some embodiments, an angiotensin (1-7) peptide or angiotensin (1-7) receptor agonist is present in an amount of or greater than about 10 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg. In some embodiments, an angiotensin (1-7) peptide or angiotensin (1-7) receptor agonist is present in an amount of or less than about 1000 mg, 950 mg, 900 mg, 850 mg, 800 mg, 750 mg, 700 mg, 650 mg, 600 mg, 550 mg, 500 mg, 450 mg, 400 mg, 350 mg, 300 mg, 250 mg, 200 mg, 150 mg, or 100 mg. In some embodiments, the therapeutically effective amount described herein is provided in one dose. In some embodiments, the therapeutically effective amount described herein is provided in one day.

In other embodiments, a therapeutically effective dosage amount may be, for example, about 0.001 mg/kg weight to 500 mg/kg weight, e.g., from about 0.001 mg/kg weight to 400 mg/kg weight, from about 0.001 mg/kg weight to 300 mg/kg weight, from about 0.001 mg/kg weight to 200 mg/kg weight, from about 0.001 mg/kg weight to 100 mg/kg weight, from about 0.001 mg/kg weight to 90 mg/kg weight, from about 0.001 mg/kg weight to 80 mg/kg weight, from about 0.001 mg/kg weight to 70 mg/kg weight, from about 0.001 mg/kg weight to 60 mg/kg weight, from about 0.001 mg/kg weight to 50 mg/kg weight, from about 0.001 mg/kg weight to 40 mg/kg weight, from about 0.001 mg/kg weight to 30 mg/kg weight, from about 0.001 mg/kg weight to 25 mg/kg weight, from about 0.001 mg/kg weight to 20 mg/kg weight, from about 0.001 mg/kg weight to 15 mg/kg weight, from about 0.001 mg/kg weight to 10 mg/kg weight. In some embodiments, the therapeutically effective amount described herein is provided in one dose. In some embodiments, the therapeutically effective amount described herein is provided in one day.

In still other embodiments, a therapeutically effective dosage amount may be, for example, about 0.001 mg/kg weight to about 1 mg/kg weight, e.g. from about 0.001 mg/kg weight to about 0.9 mg/kg weight, from about 0.001 mg/kg weight to about 0.8 mg/kg weight, from about 0.001 mg/kg weight to about 0.8 mg/kg weight, from about 0.001 mg/kg weight to about 0.7 mg/kg weight, from about 0.001 mg/kg weight to about 0.6 mg/kg weight, from about 0.001 mg/kg weight to about 0.5 mg/kg weight, from about 0.01 mg/kg weight to about 1 mg/kg weight, from about 0.01 mg/kg weight to about 0.9 mg/kg weight, from about 0.01 mg/kg weight to about 0.8 mg/kg weight, from about 0.01 mg/kg weight to about 0.7 mg/kg weight, from about 1.1 mg/kg weight to about 0.6 mg/kg weight, from about 0.01 mg/kg weight to about 0.5 mg/kg weight, from about 0.02 mg/kg weight to about 1 mg/kg weight, from about 0.02 mg/kg weight to about 0.9 mg/kg weight, from about 0.02 mg/kg weight to about 0.8 mg/kg weight, from about 0.02 mg/kg weight to about 0.7 mg/kg weight, from about 0.02 mg/kg weight to about 0.6 mg/kg weight, from about 0.02 mg/kg weight to about 0.5 mg/kg weight, from about 0.03 mg/kg weight to about 1 mg/kg weight, from about 0.03 mg/kg weight to about 0.9 mg/kg weight, from about 0.03 mg/kg weight to about 0.8 mg/kg weight, from about 0.03 mg/kg weight to about 0.7 mg/kg weight, from about 0.03 mg/kg weight to about 0.6 mg/kg weight, from about 0.03 mg/kg weight to about 0.5 mg/kg weight, from about 0.04 mg/kg weight to about 1 mg/kg weight, from about 0.04 mg/kg weight to about 0.9 mg/kg weight, from about 0.04 mg/kg weight to about 0.8 mg/kg weight, from about 0.04 mg/kg weight to about 0.7 mg/kg weight, from about 0.04 mg/kg weight to about 0.6 mg/kg weight, from about 0.04 mg/kg weight to about 0.5 mg/kg weight, from about 0.05 mg/kg weight to about 1 mg/kg weight, from about 0.05 mg/kg weight to about 0.9 mg/kg weight, from about 0.05 mg/kg weight to about 0.8 mg/kg weight, from about 0.05 mg/kg weight to about 0.7 mg/kg weight, from about 0.05 mg/kg weight to about 0.6 mg/kg weight, from about 0.05 mg/kg weight to about 0.5 mg/kg weight. In some embodiments, the therapeutically effective amount described herein is provided in one dose. In some embodiments, the therapeutically effective amount described herein is provided in one day.

In still other embodiments, a therapeutically effective dosage amount may be, for example, about 0.0001 mg/kg weight to 0.1 mg/kg weight, e.g. from about 0.0001 mg/kg weight to 0.09 mg/kg weight, from about 0.0001 mg/kg weight to 0.08 mg/kg weight, from about 0.0001 mg/kg weight to 0.07 mg/kg weight, from about 0.0001 mg/kg weight to 0.06 mg/kg weight, from about 0.0001 mg/kg weight to 0.05 mg/kg weight, from about 0.0001 mg/kg weight to about 0.04 mg/kg weight, from about 0.0001 mg/kg weight to 0.03 mg/kg weight, from about 0.0001 mg/kg weight to 0.02 mg/kg weight, from about 0.0001 mg/kg weight to 0.019 mg/kg weight, from about 0.0001 mg/kg weight to 0.018 mg/kg weight, from about 0.0001 mg/kg weight to 0.017 mg/kg weight, from about 0.0001 mg/kg weight to 0.016 mg/kg weight, from about 0.0001 mg/kg weight to 0.015 mg/kg weight, from about 0.0001 mg/kg weight to 0.014 mg/kg weight, from about 0.0001 mg/kg weight to 0.013 mg/kg weight, from about 0.0001 mg/kg weight to 0.012 mg/kg weight, from about 0.0001 mg/kg weight to 0.011 mg/kg weight, from about 0.0001 mg/kg weight to 0.01 mg/kg weight, from about 0.0001 mg/kg weight to 0.009 mg/kg weight, from about 0.0001 mg/kg weight to 0.008 mg/kg weight, from about 0.0001 mg/kg weight to 0.007 mg/kg weight, from about 0.0001 mg/kg weight to 0.006 mg/kg weight, from about 0.0001 mg/kg weight to 0.005 mg/kg weight, from about 0.0001 mg/kg weight to 0.004 mg/kg weight, from about 0.0001 mg/kg weight to 0.003 mg/kg weight, from about 0.0001 mg/kg weight to 0.002 mg/kg weight. In some embodiments, the therapeutically effective dose may be 0.0001 mg/kg weight, 0.0002 mg/kg weight, 0.0003 mg/kg weight, 0.0004 mg/kg weight, 0.0005 mg/kg weight, 0.0006 mg/kg weight, 0.0007 mg/kg weight, 0.0008 mg/kg weight, 0.0009 mg/kg weight, 0.001 mg/kg weight, 0.002 mg/kg weight, 0.003 mg/kg weight, 0.004 mg/kg weight, 0.005 mg/kg weight, 0.006 mg/kg weight, 0.007 mg/kg weight, 0.008 mg/kg weight, 0.009 mg/kg weight, 0.01 mg/kg weight, 0.02 mg/kg weight, 0.03 mg/kg weight, 0.04 mg/kg weight, 0.05 mg/kg weight, 0.06 mg/kg weight, 0.07 mg/kg weight, 0.08 mg/kg weight, 0.09 mg/kg weight, or 0.1 mg/kg weight. The effective dose for a particular individual can be varied (e.g. increased or decreased) over time, depending on the needs of the individual.

In some embodiments, the angiotensin (1-7) peptide is administered at an effective dose ranging from about 1-1,000 μg/kg/day (e.g., ranging from about 1-900 μg/kg/day, 1-800 μg/kg/day, 1-700 μg/kg/day, 1-600 μg/kg/day, 1-500 μg/kg/day, 1-400 μg/kg/day, 1-300 μg/kg/day, 1-200 μg/kg/day, 1-100 μg/kg/day, 1-90 μg/kg/day, 1-80 μg/kg/day, 1-70 μg/kg/day, 1-60 μg/kg/day, 1-50 μg/kg/day, 1-40 μg/kg/day, 1-30 μg/kg/day, 1-20 μg/kg/day, 1-10 μg/kg/day). In some embodiments, the angiotensin (1-7) peptide is administered at an effective dose ranging from about 1-500 μg/kg/day. In some embodiments, the angiotensin (1-7) peptide is administered at an effective dose ranging from about 1-100 μg/kg/day. In some embodiments, the angiotensin (1-7) peptide is administered at an effective dose ranging from about 1-60 μg/kg/day. In some embodiments, the angiotensin (1-7) peptide is administered at an effective dose selected from about 1, 2, 4, 6, 8, 10, 15, 20, 25, 30, 35, 40, 45, 50, 75, 100, 150, 200, 250, 300, 350, 400, 450, 500, 550, 600, 650, 700, 750, 800, 850, 900, 950, or 1,000 μg/kg/day.

### Dosing Schedules

Various embodiments may include differing dosing regimen. In some embodiments, the angiotensin (1-7) peptide or angiotensin (1-7) receptor agonist is administered via continuous infusion. In some embodiments, the continuous infusion is intravenous. In other embodiments, the continuous infusion is subcutaneous. Alternatively or additionally, in some embodiments, the angiotensin (1-7) peptide or angiotensin (1-7) receptor agonist is administered bimonthly, monthly, twice monthly, triweekly, biweekly, weekly, twice weekly, thrice weekly, daily, twice daily, or on another clinically desirable dosing schedule. The dosing regimen fora single subject need not be at a fixed interval, but can be varied over time, depending on the needs of the subject.

### Kits

In some embodiments, the present invention further provides kits or other articles of manufacture which contains an Ang-(1-7) peptide, an angiotensin (1-7) receptor agonist or a formulation containing the same and provides instructions for its reconstitution (if lyophilized) and/or use. Kits or other articles of manufacture may include a container, a syringe, vial and any other articles, devices or equipment useful in administration (e.g., subcutaneous, by inhalation). Suitable containers include, for example, bottles, vials, syringes (e.g., pre-filled syringes), ampules, cartridges, reservoirs, or lyo-jects. The container may be formed from a variety of materials such as glass or plastic. In some embodiments, a container is a pre-filled syringe. Suitable pre-filled syringes include, but are not limited to, borosilicate glass syringes with baked silicone coating, borosilicate glass syringes with sprayed silicone, or plastic resin syringes without silicone.

Typically, the container may hold formulations and a label on, or associated with, the container that may indicate directions for reconstitution and/or use. For example, the label may indicate that the formulation is reconstituted to concentrations as described above.

The label may further indicate that the formulation is useful or intended for, for example, subcutaneous administration. In some embodiments, a container may contain a single dose of a stable formulation containing an Ang-(1-7) peptide or angiotensin (1-7) receptor agonist. In various embodiments, a single dose of the stable formulation is present in a volume of less than about 15 ml, 10 ml, 5.0 ml, 4.0 ml, 3.5 ml, 3.0 ml, 2.5 ml, 2.0 ml, 1.5 ml, 1.0 ml, or 0.5 ml.

Alternatively, a container holding the formulation may be a multi-use vial, which allows for repeat administrations (e.g., from 2-6 administrations) of the formulation. Kits or other articles of manufacture may further include a second container comprising a suitable diluent (e.g., BWFI, saline, buffered saline). Upon mixing of the diluent and the formulation, the final protein concentration in the reconstituted formulation will generally be at least 1 mg/ml (e.g., at least 5 mg/ml, at least 10 mg/ml, at least 20 mg/ml, at least 30 mg/ml, at least 40 mg/ml, at least 50 mg/ml, at least 75 mg/ml, at least 100 mg/ml). Kits or other articles of manufacture may further include other materials desirable from a commercial and user standpoint, including other buffers, diluents, filters, needles, syringes, and package inserts with instructions for use. In some embodiments, kits or other articles of manufacture may include an instruction for self-administration.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EXAMPLES

The following examples describe some of the preferred modes of making and practicing the present invention. However, it should be understood that these examples are for illustrative purposes only and are not meant to limit the scope of the invention.

### Example 1. Correlation Between Serum Levels of Ang-(1-7) and Disease Progression in Coronavirus Infection

This example describes measurement of serum levels of Ang-(1-7) peptide in the serum of individuals suffering from lung disease, for example, as a complication of coronavirus infection, and evaluating the correlation between Ang-(1-7) levels and disease progression.

Serum samples are collected from healthy individuals, and individuals infected with coronavirus, for example, during early stage of infection (i.e. within 2 weeks of onset of symptoms) and during late stage of infection (i.e. after 2 weeks of onset of symptoms).

Levels of Ang-(1-7) are measured in the samples collected using enzyme immunoassays.

When the serum levels of Ang-(1-7) are compared between healthy individuals and individuals infected with coronavirus, if Ang-(1-7) levels are decreased in coronavirus infected individuals, it indicates downregulation of the ACE2 receptor and the alternative renin-angiotensin system.

Serum Ang-(1-7) levels are correlated with clinical disease progression and symptom severity through scoring clinical parameters such as fever, cough, and lung function tests including but not limited to spirometry and lung volume tests, pulse oximetry and chest imaging. Comparisons between early stage and late stage infections is made.

When high levels of Ang-(1-7) in serum are correlated with slower clinical disease progression and reduced symptom severity, it indicates that the proposed administration of Ang-(1-7) will have therapeutic efficacy.

### Example 2. TXA127 for the Treatment of Pneumonia Secondary to Infection by Coronavirus COVID-19

Infection by the coronavirus, COVID-19, is a worldwide epidemic, the future course of which is unknown at this time. In the more serious cases, infection leads to pneumonia which can progress to respiratory failure and death. Although the overall death rate among patients diagnosed with COVID-19 infection is estimated at around 2%, the actual death rate is probably considerably less when undiagnosed cases are included. On the other hand, most of the deaths are among hospitalized patients, so that the mortality in this population is certainly many times higher, and treatment of these patients represents a critical unmet medical need.

TXA127, which is a pharmaceutical formulation of the naturally occurring human peptide angiotensin-(1-7) (ANG-(1-7)). It is contemplated that TXA127 is the principal agonist in the Alternative Renin Angiotensin System (ARAS) pathway. ARAS counteracts the effects of the “Classical” pathway in the Renin Angiotensin System (RAS) and the balance of these two pathways is important in determining the outcome in various diseases. See FIG. 1.

The rationale for the use of TXA127 in treating COVID-19 pneumonia has at least four components:

1. The binding site for COVID-19 is the cell membrane bound enzyme ACE2, which is expressed in many tissues. ACE2 is also the attachment site for SARS and avian flu H5N1.
2. Binding of COVID-19 to ACE2 downregulates the expression of the enzyme.
3. Downregulation of ACE2 will reduce the amount of Ang-(1-7) and alter the balance between the Classical and Alternative pathways of the RAS with detrimental effects.
4. Treatment with TXA127 will restore the balance and lead to a reduction in severity of disease and mortality.

Clinical study using TXA127 to treat pneumonia secondary to infection by coronavirus COVID-19 is carried out as follows:

Approximately 100 patients with COVID-19 pneumonia are enrolled in the study. Phase 1 trial recruiting approximately 20 patients designed to confirm safety and inform the design of a Phase 2 trial. Implementation of a double blind, placebo-controlled Phase 2 trial powered to show efficacy. Implementation of a Phase 3 trial leading to regulatory approval.

Subcutaneous and/or intravenous administration of TXA127 is used to treat hospitalized patients. Oral administration is used for treatment outpatients with respiratory symptoms, which prevents their progressing to hospitalization. This protects the health care system from being overwhelmed by COVID-19 patients and reduce mortality.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

We claim:

1. A method of treating a subject infected with a coronavirus comprising administering to the subject an angiotensin (1-7) peptide or angiotensin (1-7) receptor agonist.

2. The method of claim 1, wherein the coronavirus is COVID-19, SARS or MERS.

3. The method of any one of the preceding claims, wherein the angiotensin (1-7) peptide is administered at an effective dose periodically at an administration interval such that one or more symptoms or complications of the coronavirus infection is reduced in intensity, severity, duration, or frequency or has delayed onset.

4. The method of claim 3, wherein the one or more symptoms or complications of the coronavirus infection are selected from flu-like symptoms, fever, cough, myalgia, fatigue, pneumonia, or breathing difficulties.

5. The method of claim 3, wherein the one or more symptoms or complications of the coronavirus infection comprises a cytokine storm.

6. A method of treating a lung disease or condition comprising administering to a subject in need of treatment an angiotensin (1-7) peptide or angiotensin (1-7) receptor agonist.

7. The method of claim 6, wherein the lung disease or condition is selected from bronchitis, pneumonia, pulmonary fibrosis, asthma or acute respiratory distress syndrome.

8. The method of claim 6 or 7, wherein the lung disease or condition is caused by a coronavirus infection.

9. The method of claim 8, wherein the coronavirus is COVID-19, SARS or MERS.

10. The method of claim 6 or 7, wherein the lung disease or condition is caused by avian flu H5N1.

11. The method of claim 6 or 7, wherein the lung disease or condition is caused by RSV.

12. The method of claim 6 or 7, wherein the lung disease or condition is caused by H1N1.

13. The method of any one of the preceding claims, wherein the administration is parenteral, rectal, oral, or a combination thereof.

14. The method of claim 13, wherein the parenteral administration is intravenous, subcutaneous, inhalation, intradermal, transdermal, and/or transmucosal administration.

15. The method of any one of the preceding claims, wherein the angiotensin (1-7) peptide comprises an amino acid sequence of Asp¹-Arg²-Val³-Tyr⁴-Ile⁵-His⁶-Pro⁷ (SEQ ID NO: 1) or a functional equivalent thereof.

16. The method of claim 15, wherein the angiotensin (1-7) peptide is a linear peptide.

17. The method of any one of the preceding claims, wherein the angiotensin (1-7) peptide has SEQ ID NO: 1.

18. The method of any one of claims 1-13, wherein the angiotensin (1-7) peptide comprises an amino acid sequence of Asp¹-Arg²-Val³-Ser⁴-Ile⁵-His⁶-Cys⁷ (SEQ ID NO: 2) or a functional equivalent thereof.

19. The method of any one of claims 1-13, wherein the angiotensin (1-7) peptide has an amino acid sequence of Ala¹-Arg²-Val³-Ser⁴-Ile⁵-His⁶-Cys⁷ (SEQ ID NO: 3) or a functional equivalent thereof.

20. The method of any one of the preceding claims, wherein the angiotensin (1-7) peptide comprises one or more chemical modifications to increase protease resistance, serum stability and/or bioavailability.

21. The method of claim 20, wherein the one or more chemical modifications comprise pegylation.

22. The method of any one of the preceding claims, wherein the angiotensin (1-7) peptide or angiotensin (1-7) receptor agonist is administered in combination with another treatment of the coronavirus infection or another treatment of lung disease or condition.

23. The method of any one of the preceding claims, wherein the angiotensin (1-7) peptide is administered at an effective dose ranging from about 1-1,000 μg/kg/day.

24. The method of any one of the preceding claims, wherein the angiotensin (1-7) receptor agonist is a compound with the following structure: 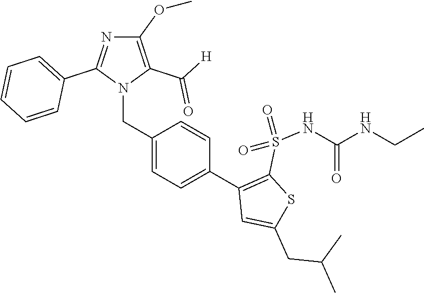 [SMILES: CCNC(=O)NS(=O)(=O)c1sc(CC(C)C)cc1-c1ccc(Cn2c(-c3ccccc3)nc(OC)c2C=O)cc1]
or a pharmaceutically acceptable salt thereof.
